(19) United States
(12) Patent Application Publication
(10) Pub. No.: US 2023/0149388 A1
(43) Pub. Date: May 18, 2023

(54) OPIOID ANTAGONIST FORMULATIONS

(71) Applicant: EMERGENT PRODUCT DEVELOPMENT GAITHERSBURG INC., Gaithersburg, MD (US)

(72) Inventors: Amir FUKHARI, Potomac, MD (US); Ryan LOUGHLIN, Babridge (IE); Vladimir SAVRANSKY, Rockville, MD (US); Andrew WALKER, Dublin (IE)

(21) Appl. No.: 17/919,574

(22) PCT Filed: Apr. 19, 2021
(86) PCT No.: PCT/US2021/027969

§ 371 Date: Oct. 18, 2022

Related U.S. Application Data
(62) Division of application No. 63/012,511, filed on Apr. 20, 2020.
(60) Provisional application No. 63/045,544, filed on Jun. 29, 2020.
(60) Provisional application No. 63/136,836, filed on Jan. 13, 2021.

(51) Int. Cl. A61K 31/485; A61K 47/02; A61K 47/18; A61K 47/12; A61M 5/20

(52) U.S. Cl. CPC A61K 31/485; A61K 47/02; A61K 47/186; A61K 47/183; A61K 47/12; A61M 5/20

(57) ABSTRACT

The present disclosure relates to pharmaceutical compositions comprising an opioid antagonist, isotonicity agent, a preservative agent, a stabilizing agent and citric acid. The pharmaceutical compositions are stable under various storage conditions. Methods of using the pharmaceutical compositions are also disclosed including methods of treatment

### GOVERNMENTAL RIGHTS

This invention was made with government support under contract number MCDC-18-03-08-004 awarded by Defense Threat Reduction Agency. The government has certain rights in the invention.

### FIELD

The present disclosure describes pharmaceutical compositions and methods for treating diseases or conditions using the same. Particularly, the present disclosure relates to opioid antagonist compositions and methods for treating opioid exposure or overdose.

### BACKGROUND

Exposure to an opioid (whether caused by an industrial accident or the drug was intentionally released) can give rise to a number of pathological conditions, including respiratory depression. Exposure to aerosolized opioid(s) can be particularly toxic. Respiratory depression can quickly result in organ and brain damage, and death. Therefore, a remedy for an opioid exposure, e.g., to an aerosolized opioid, should be fast acting, and capable of immediate use. Naloxone is an opioid receptor antagonist, able to displace opioids from opioid receptors, and thus reverse and/or inhibit the effects of an opioid exposure such as to an aerosolized opioid. Naloxone is used as an emergency treatment to reverse opioid exposure.

Naloxone can be packaged for a medical, individual or community setting. Community settings include use by an individual or anyone around the individual such as colleagues in a work environment, family members, friends, or caregivers; and by first responders such as police, fire, and life support officers. The individual can have the product stored with his/her belongings as an emergency tool. There is also a possibility of essentially weaponizing an opioid such as by aerosolizing it. For example, it could be used on a battlefield or in an urban setting. Because of the importance of opioid antagonists for the treatment of opioid exposure stable formulation of naloxone for injection is highly desirable.

### SUMMARY

Pharmaceutical dosage formulations are described herein. These dosage formulations are useful for treating, inter alia, opioid exposure or overdose, whether the opioid (for example, fentanyl or a fentanyl derivative) is voluntarily ingested or is ingested as a result of an intentional or an unintentional exposure. For example, an opioid may be intentionally or unintentionally released into the air, e.g., as suspended particles. An example of an intentional release of an opioid in the air is when an opioid is intentionally released into the air, e.g., in a warfare-like environment. An example of an unintentional release of an opioid in the air may be due to an industrial accident. Under those circumstances, the ability to self-administer or administer to another person an opioid antagonist (e.g., in a single use dosage formulation) would be an advantage.

In one embodiment, a naloxone formulation comprises between about 0.3% (w/v) and about 3.0% (w/v) naloxone or a pharmaceutically acceptable salt thereof, between about 0.3% (w/v) and about 3% (w/v) NaCl; between about 0.005% (w/v) and about 0.05% (w/v) benzalkonium chloride (“BZK”), between about 0.02% (w/v) and about 0.25% (w/v) disodium ethylenediaminetetraacetate (also known as disodium edetate and abbreviated herein as “EDTA”); and between about 0.10% (w/v) and about 1.0% (w/v) citric acid. In an embodiment, the naloxone formulation comprises between about 5 mg/mL and about 15 mg/mL naloxone, between about 5 mg/mL and about 15 mg/mL of isotonicity agent (such as NaCl), between about 0.05 mg/mL and about 0.4 mg/mL of preservative (such as BZK), between about 0.25 mg/mL and about 2 mg/mL EDTA, and between about 1 mg/mL and 10 mg/mL citric acid.

Also described herein are embodiments related to devices as well as embodiments related to devices containing the forementioned formulations. In an embodiment, there is provided a pre-loaded auto-injector device. In some embodiments, a device comprises a housing, a medicament container, and a delivery member, the medicament container disposed within the housing and defining an internal volume containing a naloxone formulation comprising: between about 0.5% (w/v) and about 2.5% (w/v) naloxone or a pharmaceutically acceptable salt thereof; between about 0.5% (w/v) and about 2.5% (w/v) NaCl; between about 0.003% (w/v) and about 0.03% (w/v) BZK; between about 0.02% (w/v) and about 0.25% (w/v) EDTA; and between about 0.10% (w/v) and about 1.0% (w/v) citric acid. In certain embodiments, these devices are configured for single use only and/or delivery of a single dose. In certain embodiments, the devices deliver two doses (“bi-dose devices”) or more than two doses, either simultaneously, or one after another, as required, to reverse the symptoms from the opioid exposure.

In certain embodiments, these methods can be used to treat, inhibit or ameliorate an opioid exposure and/or symptoms of opioid intoxication or exposure in a subject, e.g., an unconscious subject or a subject experiencing respiratory depression. In some embodiments, an opioid exposure may be due to unintentional overdose (e.g., self or doctor administered), intentional overdose (e.g., self-administration), accidental exposure, exposure due to intended exposure by another (e.g., used as a weapon). Exposure could be through any route such as injection, inhalation, or skin as well as mucosal exposure/absorption. The opioid could be any form or combination of forms such as liquid, solid, powder or aerosolized.

A naloxone formulation may be delivered by any route that delivers an effective amount of naloxone. Routes of administration include, but are not limited to, injection, intravenous, subcutaneous injection, intramuscular injection and intranasal administration. In certain embodiments, these methods involve delivering a specific volume of formulation (e.g., 50 μL, 100 μL, 200 μL, etc.) from a device into the nostril of an exposed subject. In certain embodiments, these methods involve delivering a specific volume of a formulation (e.g., 0.5 ml-2.0 ml, etc.) by injection into a subject, e.g., injecting intramuscular. In certain embodiments, only one administration of liquid is performed, while in other methods, two or more administrations are performed to inhibit, ameliorate or reverse the effects of the opioid exposure. In certain embodiments, the device is capable of delivering two doses (“bi-dose devices”), or more than two doses, either simultaneously, or one after another, as required, to treat opioid exposure in a subject in need thereof.

The naloxone formulations described herein may be used to treat humans or animals such as canines. In some embodiments, a naloxone formulation is administered to a subject (e.g., a human or an animal) prior to exposure. For example, when there is a possible or significant chance of opioid exposure such as to military, law enforcement, first responders, medical, and clean-up personnel. In some embodiments, a naloxone formulation is administered to a subject (e.g., human or animal) after known or suspected exposure to an opioid. In some embodiments, a naloxone formulation is self-administered by the exposed subject or administered by another person (e.g., colleague or medical professional) to the exposed subject. In some embodiments, administration may be made intranasally or by injection (e.g., intramuscular).

In some embodiments, the application provides for a formulation comprising, between about 0.3% (w/v) and about 3.0% (w/v) naloxone or a pharmaceutically acceptable salt thereof; between about 0.3% (w/v) and about 3% (w/v) NaCl; between about 0.005% (w/v) and about 0.05% (w/v) BZK; between about 0.02% (w/v) and about 0.25% (w/v) EDTA; and between about 0.10% (w/v) and about 1.0% (w/v) citric acid; wherein administration of about 0.15 mg/Kg to about 0.45 mg/Kg to a subject in need of provides an elimination half-life of between about 20 minutes and about 60 minutes; an AUC in plasma at between about 40 and about 70 hr*ng/mL when the AUC is measured at three times the elimination half-life or greater; a Cmax from about 30 to about 150 nanograms per milliliter; a Tmax between about 10 minutes and about 20 minutes; or bioavailability greater than about 90%; or any combination thereof.

Some embodiments of the present application provide for a method of treating an opioid exposure in a subject in need thereof, the method comprising administering to the subject about 0.15 mg/Kg to about 0.45 mg/Kg of naloxone from a formulation comprising between about 0.3% (w/v) and about 3.0% (w/v) naloxone or a pharmaceutically acceptable salt thereof; between about 0.3% (w/v) and about 3% (w/v) NaCl; between about 0.005% (w/v) and about 0.05% (w/v) BZK; between about 0.02% (w/v) and about 0.25% (w/v) EDTA; and between about 0.10% (w/v) and about 1.0% (w/v) citric acid; wherein said administration provides: an elimination half-life of between about 20 minutes and about 60 minutes; an AUC in plasma at between about 40 and about 70 hr*ng/mL when the AUC is measured at three times the elimination half-life or greater; a Cmax from about 30 to about 150 nanograms per milliliter; a Tmax between about 10 minutes and about 20 minutes; a bioavailability greater than about 90%; ora combination thereof. In some embodiments, the administration comprises an auto-injector device comprising a housing, a medicament container, and a delivery member, wherein the medicament container is disposed within the housing and defines an internal volume containing the formulation.

In some embodiments, the administration provides an elimination half-life of between about 20 minutes and about 60 minutes; and/or an AUC in plasma at between about 40 and about 70 hr*ng/mL when the AUC is measured at three times the elimination half-life or greater. In other embodiments, the administration provides an elimination half-life of between about 20 minutes and about 60 minutes; and/or a Cmax from about 30 to about 150 nanograms per milliliter. In a further embodiment, the administration provides an elimination half-life of between about 20 minutes and about 60 minutes; and/or a Tmax between about 10 minutes and about 20 minutes. In some further embodiments, the administration provides an AUC in plasma at between about 40 and about 70 hr*ng/mL when the AUC is measured at three times the elimination half-life or greater; and/or a Cmax from about 30 to about 150 nanograms per milliliter. In another embodiments, the administration provides an AUC in plasma at between about 40 and about 70 hr*ng/mL when the AUC is measured at three times the elimination half-life or greater; and/or a Tmax between about 10 minutes and about 20 minutes. In another embodiment, the administration provides a Cmax from about 30 to about 150 nanograms per milliliter; and/or a Tmax between about 10 minutes and about 20 minutes. In another embodiment, the administration provides an elimination half-life of between about 20 minutes and about 60 minutes; and/or a bioavailability greater than about 90%. In another embodiment, the administration provides an AUC in plasma at between about 40 and about 70 hr*ng/mL when the AUC is measured at three times the elimination half-life or greater; and/or a bioavailability greater than about 90%. In another embodiment, the administration provides a Cmax from about 30 to about 150 nanograms per milliliter; and/or a bioavailability greater than about 90%. In another embodiment, the administration provides a Tmax between about 10 minutes and about 20 minutes; and/or or a bioavailability greater than about 90%.

In some embodiments, the formulation has an osmolality between about 250 mOsm and 500 mOsm. In another embodiment, the formulation has a pH between about 3 and about 7, between about 3 and about 6, between about 3 and about 5, or between about 3 and about 4. In another embodiment, the formulation does not comprise a methylparaben, a propylparaben, or combinations thereof.

In some embodiments, the subject is administered a dose between about 8 mg and about 12 mg.

In some embodiments, the method comprises administering to the subject about 0.16 mg/Kg of naloxone from a formulation comprising about 1.0% (w/v) naloxone or a pharmaceutically acceptable salt thereof; about 0.9% (w/v) NaCl; about 0.02% (w/v) BZK; about 0.1% (w/v) EDTA; and about 0.5% (w/v) citric acid.

In some embodiments, the method comprises administering to the subject about 0.16 mg/Kg of naloxone from a formulation comprising about 1.0% (w/v) naloxone or a pharmaceutically acceptable salt thereof; about 0.9% (w/v) NaCl; about 0.01% (w/v) BZK; about 0.1% (w/v) EDTA; and about 0.5% (w/v) citric acid. In other embodiments, the subject is administered about 10 mg naloxone. In other embodiments, the subject is a human.

In some embodiments, the method comprises administering to the subject about 0.3 mg/Kg of naloxone from a formulation comprising: about 1.0% (w/v) naloxone or a pharmaceutically acceptable salt thereof; about 0.02% (w/v) BZK; about 0.1% (w/v) EDTA; and about 0.5% (w/v) citric acid.

In some embodiments, the method comprises administering to the subject about 0.3 mg/Kg of naloxone from a formulation comprising about 1.0% (w/v) naloxone or a pharmaceutically acceptable salt thereof; about 0.9% (w/v) NaCl; about 0.01% (w/v) BZK; about 0.1% (w/v) EDTA; and about 0.5% (w/v) citric acid. In other embodiments, the subject is a canine.

Further areas of applicability will become apparent from the description provided herein. The description and specific examples in this summary are intended for purposes of illustration only and are not intended to limit the scope of the present disclosure.

### DETAILED DESCRIPTION

The following description is merely exemplary in nature and is not intended to limit the present disclosure, application or uses.

### DESCRIPTION OF DRAWINGS

FIG. 1 shows plasma naloxone Concentration-Time Data following a Single IV or IM Injection to Male Dogs [Mean+SE; n=6].

FIG. 2 shows plasma naloxone Concentration-Time Data following a Single IV or IM Injection to Male Dogs [Mean+SE; n≥4].

### A. Definitions

As used herein, an “opioid antagonist” is a compound that counteracts the effects of opioid binding to an opioid receptor. Non-limiting examples of opioid antagonists include naloxone, or a pharmaceutically acceptable salt and/or solvate thereof (e.g., naloxone HCl or naloxone HCl.2H₂O).

As used herein, “stability,” “stable,” and similar terms connoting stability of a formulation refer to the ability of a formulation to tolerate storage at a particular temperature without significantly losing essential elements of the solution (e.g., via decomposition or precipitation) as evidenced by using analytical tools or visual observation. An analytical tool (e.g., gas or liquid chromatography, spectroscopy, etc.) or functional assay may be used to determine decomposition or inactivation of a particular ingredient or the formation of a particular impurity (which can often be traced back to the ingredient from which it originated).

As used herein, an “isotonicity agent” is an additive that is added to a solution to adjust its tonicity. Non-limiting examples of isotonicity agents include NaCl, KCl, CaCl₂, MgCl₂, NaBr, KBr, CaBr₂, MgBr₂, dextrose, glycerin, and mannitol.

As used herein, a “stabilizing agent” is an additive that is added to a solution to prevent the degradation of another agent. Non-limiting examples of stabilizing agents include calcium, sodium, and disodium ethylenediaminetetraacetic acid (EDTA), ethylene glycol-bis(β-aminoethyl ether)-N,N,N′,N′-tetraacetic acid (EGTA), sorbitol, dimercaptosuccinic acid (DMSA), calcium versetamide Na, calteridol, and diethylenetriaminepentaacetic acid (DTPA).

As used herein, a “preservative” is an additive that is added to a solution to inhibit the growth of a biological contaminant (e.g., bacteria or fungus), or to inhibit the chemical degradation of components of the formulation. Non-limiting examples of preservatives include quaternary ammonium compounds (e.g., BZK), alkyl parabens, citric acid, and alcohols. As used herein, “preservative” does not include glycols (e.g., propylene glycol and polyethylene glycol).

As used here, “alkylparaben,” alkyl paraben,” “alkylparabens,” and “alkyl parabens” are interchangeably used to indicate a pharmaceutical additive comprising one or more of methylparaben, ethylparaben, n-propylparaben, iso-propylparaben, n-butylparaben, iso-butylparaben, tert-butylparaben, n-pentylparaben, tert-pentylparaben, neo-pentylparaben, 1,1-dimethylpropylparaben, 2,2-dimethylpropylparaben, 3-methylbutylparaben, 1-methylbutylparaben, 1-ethylpropylparaben, 1,2-dimethylpropylparaben, or 2-methylbutylparaben.

As used herein, a “subject” refers to an animal. Typically, the animal is a mammal that would benefit from treatment with a formulation described herein. Particular examples include primates (e.g., humans), dogs, cats, horses, cows, pigs, and sheep. Individuals and subjects are also subjects herein.

As used herein, “subject in need thereof” means a subject in need of treatment, for example, because of actual, suspected or possible/likely future exposure to an opioid. In some embodiments, a subject has one or more indications or symptoms related to opioid exposure or toxicity.

As used herein, “administering” means providing a formulation to a subject. This concept includes when administered by anyone such as a medical professional, the subject himself or a non-medical professional.

The term “fentanyl derivative” as used herein refers to a molecule of Formula (I)

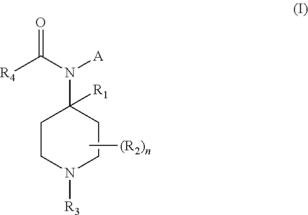

wherein A is aryl or heteroaryl optionally substituted with halo, C₁-C₃ alkyl, or C₁-C₃ alkoxy,

R₁ and R₂ are each independently selected from the group consisting of phenyl, C₁-C₃ alkyl, C₂-C₃ alkenyl, C₁-C₃ alkoxyalkyl, or C₁-C₃ alkoxy, and —COOCH₃,

R₃ is C₁-C₃ alkyl or hydroxyethyl, optionally substituted with —COOCH₃, aryl, or heteroaryl optionally substituted with both C₁-C₃ alkyl and ═O,

R₄ is C₁-C₄ alkyl, C₂-C₃ alkenyl, C₁-C₃ alkoxy, C₁-C₃ alkoxyalkyl, cycloalkyl, or heteroaryl, and

n is 1, 2, or 3.

Non-limiting examples of fentanyl derivatives are disclosed in WO 2017/049181 to Keegan et al.

As used here, the term “about” means that the stated number can vary from that value by ±10%. Where the term defines quantity (such as weight), the term means the quantity can vary by ±10%. For example, about 5% (w/w or w/v) means between 4.5% and 5.5% (w/w or w/v). Further, about 1 mg/mL means between 0.9 mg/mL and 1.1 mg/mL. Where the term defines a temperature, the stated temperature can vary by ±10%. For example, about 80° C. means between 72° C. and 88° C. Where the term defines time, the term means the stated time can vary by ±10%. For example, about 1 hour means between 0.9 and 1.1 hours.

All mentioned documents are incorporated by reference as if herein written. When introducing elements of the present disclosure or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations.

### B: Opioid Antagonists Formulations

Formulations described herein comprise an opioid antagonist (such as naloxone), an isotonicity agent, a preservative, and a stabilizing agent. In certain embodiments, the isotonicity agent is present in an amount sufficient to achieve an osmolality between about 250 mOsm and 500 mOsm. In certain embodiments, the isotonicity agent is NaCl, KCl, CaCl₂, or MgCl₂. In certain embodiments, the isotonicity agent comprises NaCl. In certain embodiments, the preservative may be a quaternary ammonium compound (such as BZK), alkyl parabens, citric acid, and alcohols. In certain embodiments, the preservative comprises BZK and citric acid. In certain embodiments, the stabilizing agent comprises EDTA.

In some embodiments, the formulation does not comprise a methylparaben, a propylparaben, or combinations thereof. In some embodiments, the formulation does not comprise an alkyl paraben. In certain embodiments, the preservative does not comprise an alkyl paraben. In some embodiments, the formulation does not comprise an alkyl paraben to an extent that would affect the stability of the formulation or its efficacy otherwise. In certain embodiments, the total amount of methylparaben, propylparaben or alkyl parabens present in the formulation is no greater than about 0.001% (w/v), about 0.002% (w/v), about 0.003% (w/v), about 0.004% (w/v), about 0.005% (w/v), about 0.006% (w/v), about 0.007% (w/v), about 0.008% (w/v), about 0.009% (w/v), about 0.001% (w/v), about 0.010% (w/v), about 0.011% (w/v), about 0.012% (w/v), about 0.013% (w/v), about 0.014% (w/v), about 0.015% (w/v), about 0.016% (w/v), about 0.017% (w/v), about 0.018% (w/v), about 0.019% (w/v), or about 0.020% (w/v).

In an embodiment, the opioid antagonist is naloxone, a pharmaceutically acceptable salt thereof, or a solvate thereof. In certain embodiments, naloxone is provided as a free base. In certain embodiments, naloxone is provided as a salt (e.g., a hydrochloride or an acetate salt). In certain embodiments, naloxone is provided as a solvate, or a solvate of a salt (e.g., naloxone hydrochloride dihydrate).

In certain embodiments, naloxone formulations described herein are used or administered at a dose between about 5 mg and about 15 mg, between about 6 mg and about 14 mg, between about 7 mg and about 13 mg, between about 8 mg and about 12 mg, or between about 9 mg and about 11 mg. For example, naloxone is used or administered at a dose of about 5 mg, about 6 mg, about 7 mg, about 8 mg, about 9 mg, about 10 mg, about 11 mg, about 12 mg, about 13 mg, about 14 mg, or about 15 mg.

In some embodiments the formulation of naloxone comprises between about 0.2% (w/v) and about 3% (w/v) naloxone, for example between about 0.5% (w/v) and about 2% (w/v), between about 0.7% (w/v) and about 1.8% (w/v), between about 0.8% (w/v) and about 1.7% (w/v), between about 0.8% (w/v) and about 1.5% (w/v), between about 0.8% (w/v) and about 1.4% (w/v), between about 0.9% (w/v) and about 1.2% (w/v), or between about 0.9% (w/v) and about 1.1% (w/v). In certain embodiments, the formulation comprises about 0.5% (w/v), about 0.6% (w/v), about 0.7% (w/v), about 0.8% (w/v), about 0.9% (w/v), about 1.0% (w/v), about 1.1% (w/v), about 1.2% (w/v), about 1.3% (w/v), about 1.4% (w/v), or about 1.5% (w/v) naloxone.

In certain embodiments, formulations of naloxone described herein may also comprise citric acid at about 0.10% (w/v), about 0.15% (w/v), about 0.18% (w/v), about 0.20% (w/v), about 0.25% (w/v), about 0.30% (w/v), about 0.35% (w/v), about 0.40% (w/v), about 0.45% (w/v), about 0.48% (w/v), about 0.50% (w/v), about 0.55% (w/v), about 0.60% (w/v), about 0.65% (w/v), about 0.70% (w/v), about 0.75% (w/v), about 0.80% (w/v), about 0.85% (w/v), about 0.90% (w/v), about 0.95% (w/v), or about 1.00% (w/v) citric acid. In some embodiments, the formulation comprises about 0.10% (w/v) to about 1.00% (w/v), about 0.10% (w/v) to about 0.90% (w/v), about 0.10% (w/v) to about 0.80% (w/v), about 0.10% (w/v) to about 0.75% (w/v), about 0.10% (w/v) to about 0.70% (w/v), about 0.10% (w/v) to about 0.65% (w/v), about 0.10% (w/v) to about 0.60% (w/v), about 0.10% (w/v) to about 0.55% (w/v), or about 0.10% (w/v) to about 0.50% (w/v) citric acid. In some embodiments, the formulation comprises about 0.20% (w/v) to about 0.80% (w/v), comprises about 0.30% (w/v) to about 0.70% (w/v), comprises about 0.40% (w/v) to about 0.50% (w/v), comprises about 0.45% (w/v) to about 0.45% (w/v), comprises about 0.46% (w/v) to about 0.5% (w/v), or comprises about 0.47% (w/v) to about 0.49% (w/v) citric acid.

In certain embodiments, a formulation of naloxone described herein may also comprise between about 0.1% (w/v) and about 2% (w/v) NaCl, for example between about 0.2% (w/v) and about 1.9% (w/v), between about 0.4% (w/v) and about 1.7% (w/v), between about 0.6% (w/v) and about 1.4% (w/v), between about 0.7% (w/v) and about 1.3% (w/v), between about 0.8% (w/v) and about 1.3% (w/v), between about 0.8% (w/v) and about 1.2% (w/v), between about 0.8% (w/v) and about 1.1% (w/v), between about 0.8% (w/v) and about 1.0% (w/v), between about 0.8% (w/v) and about 0.9% (w/v), between about 0.8% (w/v) and about 0.85% (w/v), or between about 0.85% (w/v) and about 0.95% (w/v). In certain embodiments, a formulation may contain about 0.5% (w/v), about 0.6% (w/v), about 0.7% (w/v), about 0.83% (w/v), about 0.85% (w/v), about 0.88% (w/v), about 0.92% (w/v), about 0.94% (w/v), about 0.95% (w/v), about 0.96% (w/v), about 0.98% (w/v), about 1.00% (w/v), about 1.05% (w/v), about 1.10% (w/v), about 1.15% (w/v), or about 1.20% (w/v) NaCl. In certain embodiments, a formulation may comprise about 0.80% (w/v), about 0.82% (w/v), about 0.84% (w/v),about 0.86% (w/v), about 0.87% (w/v), about 0.90% (w/v), about 0.91% (w/v), about 0.93% (w/v), about 0.94% (w/v), about 0.97% (w/v), about 0.99% (w/v), or about 1.00% (w/v) NaCl.

In certain embodiments, a formulation described herein comprises about 0.01% (w/v) to about 0.5% (w/v) EDTA, for example about 0.03% (w/v) to about 0.2% (w/v), about 0.03% (w/v) to about 0.07% (w/v), about 0.04% (w/v) to about 0.06% (w/v), about 0.045% (w/v) to about 0.055% (w/v), about 0.07% (w/v) to about 0.13% (w/v), about 0.08% (w/v) to about 0.12% (w/v), about 0.09% (w/v) to about 0.11% (w/v), about 0.095% (w/v) to about 0.105% (w/v), about 0.03% (w/v) to about 0.13% (w/v), about 0.05% (w/v) to about 0.1% (w/v), about 0.06% (w/v) to about 0.09% (w/v), or about 0.07% (w/v) to about 0.08% (w/v) EDTA. In certain embodiments, a formulation may contain about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.10% (w/v), about 0.11% (w/v), about 0.12% (w/v), about 0.13% (w/v), about 0.14% (w/v), about 0.15% (w/v), about 0.16% (w/v), about 0.17% (w/v), about 0.18% (w/v), about 0.19% (w/v), or about 0.20% (w/v) EDTA. In certain embodiments, a formulation may comprise about 0.01% (w/v) to about 0.15% (w/v) EDTA. In certain embodiments, a formulation may comprise about 0.02% (w/v) to about 0.15% (w/v), about 0.02% (w/v) to about 0.14% (w/v), about 0.03% (w/v) to about 0.13% (w/v), about 0.04% (w/v) to about 0.12% (w/v), or about 0.05% (w/v) to about 0.10% (w/v) EDTA.

In certain embodiments, a formulation of naloxone described herein may also comprise BZK. In certain embodiments, for example, formulations may comprise between about 0.005% (w/v) and about 0.10% (w/v) BZK, for example between about 0.005% (w/v) and about 0.015% (w/v), between about 0.006% (w/v) and about 0.014% (w/v), between about 0.007% (w/v) and about 0.013% (w/v), between about 0.008% (w/v) and about 0.012% (w/v), between about 0.009% (w/v) and about 0.011% (w/v), between about 0.0095% (w/v) and about 0.0105% (w/v), between about 0.005% (w/v) and about 0.03% (w/v), between about 0.01% (w/v) and about 0.02% (w/v), between about 0.012% (w/v) and about 0.018% (w/v), between about 0.014% (w/v) and about 0.016% (w/v), between about 0.016% (w/v) and about 0.024% (w/v), between about 0.017% (w/v) and about 0.023% (w/v), between about 0.018% (w/v) and about 0.022% (w/v), between about 0.019% (w/v) and about 0.021% (w/v), or between about 0.0195% (w/v) and about 0.0205% (w/v) BZK. In certain embodiments, a formulation may contain about 0.005% (w/v), about 0.006% (w/v), about 0.007% (w/v), about 0.008% (w/v), about 0.009% (w/v), about 0.01% (w/v), about 0.011% (w/v), about 0.012% (w/v), about 0.013% (w/v), about 0.014% (w/v), about 0.015% (w/v), about 0.016% (w/v), about 0.017% (w/v), about 0.018% (w/v), about 0.019% (w/v), about 0.020% (w/v), about 0.021% (w/v), about 0.022% (w/v), about 0.023% (w/v), about 0.024% (w/v), about 0.025% (w/v), about 0.026% (w/v), about 0.027% (w/v), about 0.028% (w/v), about 0.029% (w/v), or about 0.03% (w/v) BZK. In certain embodiments, a formulation may contain about 0.010% (w/v), 0.012% (w/v), about 0.014% (w/v), about 0.016% (w/v), about 0.018% (w/v), or about 0.02% (w/v) BZK.

In certain embodiments, formulations contain about 0.005% (w/v) to about 0.030% (w/v), about 0.006% (w/v) to about 0.030% (w/v), about 0.007% (w/v) to about 0.030% (w/v), about 0.008% (w/v) to about 0.030% (w/v), about 0.009% (w/v) to about 0.030% (w/v), about 0.01% (w/v) to about 0.030% (w/v), about 0.01% (w/v) to about 0.028% (w/v), about 0.01% (w/v) to about 0.027% (w/v), about 0.01% (w/v) to about 0.026% (w/v), about 0.01% (w/v) to about 0.025% (w/v), about 0.01% (w/v) to about 0.024% (w/v), about 0.01% (w/v) to about 0.023% (w/v), about 0.01% (w/v) to about 0.022% (w/v), about 0.01% (w/v) to about 0.021% (w/v), or about 0.01% (w/v) to about 0.020% (w/v) BZK.

The pH of a formulation described herein should be appropriate to ensure that the naloxone delivered into the body via an auto-injector can be absorbed into the blood. The formulations described herein will have a pH between about 3 and about 7, for example between about 3.0 and about 5.0. The formulations described herein will have a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0. In certain embodiments, the formulations have a pH about 3.0 to about 4.5, about 3.0 to about 4.0, about 3.1 to about 3.9, about 3.2 to about 3.8, about 3.3 to about 3,7, or about 3.4 to about 3.6. Where the pH of the formulation lies outside of the desired range once all of the ingredients are dissolved in the formulation, an appropriate quantity of acid, base, or buffer can be added as necessary to adjust the pH before bringing the formulation to its final volume. In certain embodiments, the acid is an inorganic acid. In certain embodiments, the acid is HCl or H₂SO₄. In certain embodiments the base is NaOH or KOH. In certain embodiments the buffer is phosphate buffer, acetate buffer, potassium hydrogen phthalate buffer, glycine buffer, disodium hydrogen phthalate buffer, sodium dihydrogen orthophosphate buffer, carbonate buffer, benzoate buffer, hydrobromic acid buffer, lactic acid buffer, tartaric acid buffer, or citrate buffer.

In certain embodiments, the osmolality of a formulation is about 250 mOsm to about 500 mOsm, about 300 mOsm to about 450 mOsm, about 325 mOsm to about 425 mOsm, about 350 mOsm to about 400 mOsm, about 325 mOsm to about 375 mOsm, about 350 mOsm to about 360 mOsm, about 380 mOsm to about 390 mOsm, about 410 mOsm to about 420 mOsm, about 285 mOsm to about 295 mOsm, about 325 mOsm to about 335 mOsm, or about 360 mOsm to about 370 mOsm. In certain embodiments, the osmolality of a formulation is about 386 mOsm, about 332 mOsm, about 355 mOsm, about 417 mOsm, about 288 mOsm, or about 367 mOsm. In certain embodiments, the naloxone formulations described herein may also comprise impurities. As used herein, an “impurity” is any detectable chemical species whose presence in the formulation was not intended by the manufacturer. An “impurity” may be noxious, but it may also be innocuous, and the detection of an impurity does not necessarily indicate that the formulation is toxic or otherwise unsuitable for administration to a subject. In certain embodiments, the incidence of impurities increases during storage of the formulations. Certain impurities can be detected by reverse-phase high pressure liquid chromatography (RP-HPLC) that have relative retention times of less than one. Certain impurities can also be detected by RP-HPLC that have relative retention times greater than one. An example RP-HPLC method utilizes a mobile phase (25 mM sodium phosphate at pH 6.8:acetonitrile), with a flow rate at 0.8 mL/min, ultra-violet (UV) detection at 229 nm, 10 μL injection volume, and a C6-phenyl column with a column temperature of 40° C. for a run time of 20 minutes.

In some embodiments, the opioid is fentanyl, or a fentanyl derivative. In various embodiments, the fentanyl derivative is a compound of Formula (I). In certain embodiments, the fentanyl derivative may be fentanyl, 3-allylfentanyl, 3-methylfentanyl, 4-fluorobutyrfentanyl, p-fluoroisobutyrfentanyl, 4-phenylfentanyl, acrylfentanyl, α-methylbutyrfentanyl, α-methylthiofentanyl, alfentanyl, β-hydroxyfentanyl, β-methylfentanyl, brifentanyl, cyclopentylfentanyl, furanylfentanyl, lofentanyl, methoxyacetylfentanyl, ocfentanyl, R-30490, sufentanyl, thiofentanyl, valerylfentanyl, 2,5-dimethylfentanyl, 3-methylbutyrfentanyl, 3-methylthiofentanyl, p-chloroisobutyrfentanyl, 4-fluorofentanyl, 4-methoxybutyrfentanyl, α-methylacetylfentanyl, a-methylfentanyl, acetylfentanyl, benzylfentanyl, β-hydroxythiofentanyl, butyrfentanyl, carfentanyl, isobutyrfentanyl, furanylethylfentanyl, n-methylcarfentanyl, mirfentanyl, ohmefentanyl, remifentanyl, thenylfentanyl, or trefentanyl.

In some embodiments, said subject is in a lying, supine, or recovery position. In some embodiments, said subject is in a lying position. In some embodiments, said subject is in a supine position. In some embodiments, said subject is in a recovery position. The recovery position is a position of the human body in which a subject lies on his/her side, with a leg or knee out in front (e.g., to prevent rolling onto his/her stomach) and at least one hand supporting the head (e.g., to elevate the face to facilitate breathing and prevent inhalation of vomit).

### C. Devices

Also provided herein are auto-injector devices containing the formulations described herein. In some embodiments, the auto-injector devices contain single-dose formulations. In some embodiments, the auto-injector devises are single use. Non-limiting examples of devices suitable for use in delivering the formulations described herein can be found in U.S. Pat. Nos. 6,743,203, 7,449,012, 7,611,491, 8,647,306, US Patent Publication US2019-0009025, U.S. Pat. Nos. 10,143,792, 10,220,158, and 10,342,924, each of which is incorporated by reference in its entirety.

### D. Methods of Use

In certain embodiments, formulations and devices described herein can be used to treat opioid exposure and related symptoms. One advantage of the formulations and devices described herein is that they are acceptable for injection of naloxone. In some embodiments, a naloxone formulation described herein is administered by injection, for example, via a syringe or an autoinjector. Injection of naloxone using an auto-injector has certain advantages over traditional injection, in that auto-injector administration requires less, or no, medical training and hence can be used in a variety of settings including community settings. This is in contrast to traditional injections administered at healthcare facilities or by healthcare professionals which may require medical training and/or actions to prepare the injection. Hence delivery via auto-injection has substantial advantages, particularly when used in community settings or in response to an exposure that requires immediate action, for example in the high stress situation when a person needs immediate administration of naloxone because of aerial release of an opioid. Speed and simplicity of administration clearly also matters when looking to reverse an exposure or contamination in order to prevent organ damage or death which can arise with respiratory depression. In addition, administration via an auto-injector minimizes the likelihood of accidental needle sticks and the corresponding danger of blood-borne infection.

Some methods disclosed herein will achieve acceptable serum concentration of the opioid antagonist. For example, in certain embodiments the plasma concentration versus time curve of opioid antagonist (e.g., naloxone) in the subject has a tmax of less than 30 minutes. In certain embodiments, the subject experiences a geometric mean naloxone Cmax not less than about 3 ng/mL following auto-injection. In certain embodiments, the subject experiences a plasma naloxone concentration such that the geometric mean of area under a plasma concentration versus time curve (AUC_{0-∞}) is not less than about 8 hr*ng/mL when time is extrapolated to infinity.

### E. Exemplary Embodiments

The present disclosure further provides the following non-limiting embodiments.

Embodiment 1. A formulation comprising between about 0.3% (w/v) and about 3.0% (w/v) naloxone or a pharmaceutically acceptable salt thereof, between about 0.3% (w/v) and about 3% (w/v) NaCl, between about 0.005% (w/v) and about 0.05% (w/v) BZK, between about 0.02% (w/v) and about 0.25% (w/v) EDTA, and between about 0.10% (w/v) and about 1.0% (w/v) citric acid.

Embodiment 2. The formulation of embodiment 1, comprising between about 0.7% (w/v) and about 1.5% (w/v) naloxone or a pharmaceutically acceptable salt thereof, between about 0.5% (w/v) and about 1.5% (w/v) NaCl, between about 0.005% (w/v) and about 0.03% (w/v) BZK, between about 0.05% (w/v) and about 0.10% (w/v) EDTA, and between about 0.20% (w/v) and about 0.50% (w/v) citric acid.

Embodiment 3. The formulation of embodiment 2, wherein the formulation comprises about 1% (w/v) naloxone or a pharmaceutically acceptable salt thereof.

Embodiment 4. The formulation of any one of embodiments 1-3, wherein the NaCl is present in a concentration between about 0.7% (w/v) and about 1.4% (w/v).

Embodiment 5. The formulation of any one of embodiments 1-3, wherein the NaCl is present in a concentration between about 0.8% (w/v) and about 1.2% (w/v).

Embodiment 6. The formulation of any one of embodiments 1-3, wherein the NaCl is present in a concentration about 0.8% (w/v), about 0.9% (w/v), or about 1.0% (w/v).

Embodiment 7. The formulation of any one of embodiments 1-6, wherein BZK is present in an amount between about 0.01% (w/v) and about 0.02% (w/v).

Embodiment 8. The formulation of any one of embodiments 1-6 wherein BZK is present in an amount about 0.008% (w/v), 0.009% (w/v), 0.010% (w/v), about 0.012% (w/v), about 0.014% (w/v), about 0.016% (w/v), about 0.018% (w/v), about 0.02% (w/v), about 0.021% (w/v), or about 0.022% (w/v).

Embodiment 9. The formulation of any one of embodiments 1-8, wherein the formulation has an osmolality between about 250 mOsm and 500 mOsm.

Embodiment 10. The formulation of any one of embodiments 1-9, wherein the formulation has a pH between about 3 and about 7, between about 3 and about 6, between about 3 and about 5, or between about 3 and about 4.

Embodiment 11. The formulation of any one of embodiments 1-9, wherein the formulation has a pH about 3.5.

Embodiment 12. The formulation of any one of embodiments 1-11, wherein the EDTA is present about 0.03%, about 0.04%, about 0.05% about 0.06%, about 0.07%, about 0.08%, about 0.09%, about 0.10%, about 0.11%, or about 0.12% (w/v).

Embodiment 13: The formulation of any one of embodiments 1-12, wherein the formulation does not comprise a methylparaben, a propylparaben, or combinations thereof.

Embodiment 14. The formulation of any one of embodiments 1-12, wherein the formulation does not comprise alkylparabens.

Embodiment 15. The formulation of any one of embodiments 1-12, wherein the total amount of alkylparabens present in the formulation is no greater than about 0.001% (w/v).

Embodiment 16. An auto-injector device, wherein the device comprises a housing, a medicament container, and a delivery member, wherein the medicament container is disposed within the housing and defines an internal volume containing the formulation of any one of embodiments 1-15.

Embodiment 17. A method of treating an opioid exposure in a subject in need thereof, the method comprising administering the formulation of any one of embodiments 1-15, to a subject.

Embodiment 18. The method of embodiment 16, wherein the administering comprises injecting the subject with the formulation.

Embodiment 19. The method of embodiment 16, wherein the administration comprises injecting the subject intramuscularly with the formulation.

Embodiment 20. The method of embodiment 16, wherein the administration comprises injecting the subject subcutaneously with the formulation.

Embodiment 21. The method of any one of embodiments 16-19, wherein the administration is performed with an autoinjector.

Embodiment 22. A formulation comprising between about 0.3% (w/v) and about 3.0% (w/v) naloxone or a pharmaceutically acceptable salt thereof, between about 0.3% (w/v) and about 3% (w/v) NaCl, between about 0.005% (w/v) and about 0.05% (w/v) BZK, between about 0.02% (w/v) and about 0.25% (w/v) EDTA, and between about 0.10% (w/v) and about 1.0% (w/v) citric acid; wherein administration of about 0.15 mg/Kg to about 0.45 mg/Kg to a subject in need of provides an elimination half-life of between about 20 minutes and about 60 minutes; an AUC in plasma at between about 40 and about 70 hr*ng/mL when the AUC is measured at three times the elimination half-life or greater; a Cmax from about 30 to about 150 nanograms per milliliter; a Tmax between about 10 minutes and about 20 minutes; or bioavailability greater than about 90%; or any combination thereof.

Embodiment 23. A method of treating an opioid exposure in a subject in need thereof, the method comprising administering to the subject about 0.15 mg/Kg to about 0.45 mg/Kg of naloxone from a formulation comprising between about 0.3% (w/v) and about 3.0% (w/v) naloxone or a pharmaceutically acceptable salt thereof; between about 0.3% (w/v) and about 3% (w/v) NaCl; between about 0.005% (w/v) and about 0.05% (w/v) BZK; between about 0.02% (w/v) and about 0.25% (w/v) EDTA; and between about 0.10% (w/v) and about 1.0% (w/v) citric acid; wherein administration of about 0.15 mg/Kg to about 0.45 mg/Kg of naloxone provides an elimination half-life of between about 20 minutes and about 60 minutes; an AUC in plasma at between about 40 and about 70 hr*ng/mL when the AUC is measured at three times the elimination half-life or greater; a Cmax from about 30 to about 150 nanograms per milliliter; a Tmax between about 10 minutes and about 20 minutes; a bioavailability greater than about 90%; or a combination thereof.

Embodiment 24. The method of embodiment 23, wherein said administration comprises an auto-injector device comprising a housing, a medicament container, and a delivery member, wherein the medicament container is disposed within the housing and defines an internal volume containing the formulation.

Embodiment 25. The method of embodiment 24, wherein said administration provides an elimination half-life of between about 20 minutes and about 60 minutes; and/or an AUC in plasma at between about 40 and about 70 hr*ng/mL when the AUC is measured at three times the elimination half-life or greater.

Embodiment 26. The method of embodiment 23, wherein said administration provides an elimination half-life of between about 20 minutes and about 60 minutes; and/or a Cmax from about 30 to about 150 nanograms per milliliter.

Embodiment 27. The method of embodiment 23, wherein said administration provides an elimination half-life of between about 20 minutes and about 60 minutes; and/or a Tmax between about 10 minutes and about 20 minutes.

Embodiment 28. The method of embodiment 23, wherein said administration provides an AUC in plasma at between about 40 and about 70 hr*ng/mL when the AUC is measured at three times the elimination half-life or greater; and/or a Cmax from about 30 to about 150 nanograms per milliliter.

Embodiment 29. The method of embodiment 23, wherein said administration provides an AUC in plasma at between about 40 and about 70 hr*ng/mL when the AUC is measured at three times the elimination half-life or greater; and/or a Tmax between about 10 minutes and about 20 minutes.

Embodiment 30. The method of embodiment 23, wherein said administration provides a Cmax from about 30 to about 150 nanograms per milliliter; and/or a Tmax between about 10 minutes and about 20 minutes.

Embodiment 31. The method of any of embodiments 23-31, wherein an elimination half-life of between about 20 minutes and about 60 minutes; and/or a bioavailability greater than about 90%.

Embodiment 32. The method of any of embodiments 23-31, wherein an AUC in plasma at between about 40 and about 70 hr*ng/mL when the AUC is measured at three times the elimination half-life or greater; and/or a bioavailability greater than about 90%.

Embodiment 33. The method of any of embodiments 23-31, wherein a Cmax from about 30 to about 150 nanograms per milliliter; and/or a bioavailability greater than about 90%.

Embodiment 34. The method of any of embodiments 23-31, wherein a Tmax between about 10 minutes and about 20 minutes; and/or or a bioavailability greater than about 90%.

Embodiment 35. The method of any of embodiments 23-34, wherein the formulation has an osmolality between about 250 mOsm and 500 mOsm.

Embodiment 36. The method of any of embodiments 23-35, wherein the formulation has a pH between about 3 and about 7, between about 3 and about 6, between about 3 and about 5, or between about 3 and about 4.

Embodiment 37. The method of any of embodiments 23-36, wherein the formulation does not comprise a methylparaben, a propylparaben, or combinations thereof.

Embodiment 38. The method of any of embodiments 23-37, wherein the subject is administered a dose between about 8 mg and about 12 mg.

Embodiment 39. The method of any of embodiments 23-38, wherein the method comprises administering to the subject about 0.16 mg/Kg of naloxone from a formulation comprising about 1.0% (w/v) naloxone or a pharmaceutically acceptable salt thereof; about 0.9% (w/v) NaCl; about 0.02% (w/v) BZK; about 0.1% (w/v) EDTA; and about 0.5% (w/v) citric acid.

Embodiment 40. The method of any of embodiments 23-38, wherein the method comprises administering to the subject about 0.16 mg/Kg of naloxone from a formulation comprising about 1.0% (w/v) naloxone or a pharmaceutically acceptable salt thereof; about 0.9% (w/v) NaCl; about 0.01% (w/v) BZK; about 0.1% (w/v) EDTA; and about 0.5% (w/v) citric acid.

Embodiment 41. The method of any of embodiments 23-40, wherein the subject is administered about 10 mg naloxone.

Embodiment 42. The method of any of embodiments 23-41, wherein the subject is a human.

Embodiment 43. The method of any of embodiments 23-39, wherein the method comprises administering to the subject about 0.3 mg/Kg of naloxone from a formulation comprising about 1.0% (w/v) naloxone or a pharmaceutically acceptable salt thereof; about 0.02% (w/v) BZK; about 0.1% (w/v) EDTA; and about 0.5% (w/v) citric acid.

Embodiment 44. The method of any of embodiments 23-38, wherein the method comprises administering to the subject about 0.3 mg/Kg of naloxone from a formulation comprising about 1.0% (w/v) naloxone or a pharmaceutically acceptable salt thereof; about 0.9% (w/v) NaCl; about 0.01% (w/v) BZK; about 0.1% (w/v) EDTA; and about 0.5% (w/v) citric acid.

Embodiment 45. The method of any of embodiments 43 and 44, wherein the subject is a canine.

### EXAMPLES

Without further description, it is believed that one of ordinary skill in the art can, using the preceding description and the following illustrative examples, make and use the formulations and devices described herein and practice the methods disclosed herein.

### Example 1

### Preparation of Formulations

The purity and impurity profiles for naloxone HCl were determined using HPLC analysis. Potential impurities in naloxone HCl (such as noroxymorphone, 10-ketonaloxone, 10-hydroxynaloxone and 2,2′-bisnaloxone) were measured using reverse-phase HPLC (25 mM sodium phosphate pH 6.8 as mobile phase A and acetonitrile as mobile phase B) using UV 229 nm detector, and Gemini C6-phenyl 110A column (4.6 mm×15 cm (5 μm) packing) at 40° C., a flow rate of 0.8 mL/min and the following mobile phase gradient:

| | Mobile | Mobile |
|---|---|---|
| Time | Phase % A | Phase % B |
| 0 | 60 | 40 |
| 10 | 50 | 50 |
| 13 | 40 | 60 |
| 17 | 60 | 40 |
| 20 | 60 | 40 |

An LC/MS system was used to measure A-7 naloxone.

Solution pH was measured using method described in United States Pharmacopeia.

Exemplary formulations for use in the following experiments were prepared as follows: all excipients (see, Table 1 below for the ingredients and their amounts in each formulation) were dissolved in water to achieve a volume approximately 10% less than the target volume. The pH was then adjusted to about 3.5 using HCl or NaOH as appropriate. The formulation was sonicated (if needed) for about 10 minutes to ensure complete dissolution of solid materials. Finally, the remainder of the water was added to reach target volume and its pH was verified for a second time and adjusted as necessary.

TABLE 1
Formulations 1-9

| | | Formulation | | | | | | | | |
|---|---|---|---|---|---|---|---|---|---|---|
| Component | Unit | 1 | 2 | 3 | 4 | 5 | 6 | 7 | 8 | 9 |
| Naloxone HCl | mg/mL | 10 | 10 | 10 | 10 | 10 | 10 | 10 | 10 | 10 |
| NaCI | mg/mL | 9 | 9 | 9 | 9 | 9 | 9 | 9 | 8.35 | 8.35 |
| BZK | mg/mL | 0.2 | 0.2 | 0.1 | 0.1 | 0.1 | 0.1 | 0 | 0 | 0 |
| EDTA | mg/mL | 1 | 0.5 | 1 | 0.5 | 0.5 | 0 | 0 | 0 | 0.5 |
| Citric acid | mg/mL | 4.81 | 4.81 | 4.81 | 4.81 | 1.92 | 1.92 | 1.92 | 0 | 0 |
| Methylparabens | mg/mL | 0 | 0 | 0 | 0 | 0 | 0 | 0 | 1.8 | 1.8 |
| Propylparabens | mg/mL | 0 | 0 | 0 | 0 | 0 | 0 | 0 | 0.2 | 0.2 |
| pH | N/A | 3.5 | 3.5 | 3.5 | 3.5 | 3.5 | 3.5 | 3.5 | 4.0 | 4.0 |

| | | |
|---|---|---|
| Purified H₂O | N/A | Adjust volume to 1 mL |

A larger scale formulation suitable for preparing formulations 1-9 may also be prepared similar to the examples provided in the stability study (see Example 2 below).

### Example 2

### Stability Studies

At the initial time-point, each formulation was tested in duplicate. For each temperature and time-point thereafter, four 10 mL vials were prepared and stored at 2-8° C. and 25° C. at 60% relative humidity (RH), 40° C. at 75% RH and 50° C. for up to six months. Three of the four vials (per stability time/test point) were analyzed to provide a data point in triplicate. The fourth vial was used in the case of a deviation which would call into question the integrity of an analytical result obtained (i.e. dropped/spilled vial).

The samples were prepared as following. For each of formulations 1-5, approximately 600 mL of Type 1 water was added into a 1 liter flask containing a clean magnetic stirrer. While stirring, 9.00 g of sodium chloride (NaCl) added and dissolved. Once the NaCl was dissolved, an appropriate amount of benzalkonium chloride 10% (BZK) was added. Once the BZK was dissolved, an appropriate amount of disodium EDTA (EDTA) was added. Once the EDTA was dissolved, an appropriate amount of citric acid was added. Once the citric acid was dissolved, 10.99 g of naloxone HCl dihydrate was added. The pH of the solution was adjusted to 3.5 using 1 N hydrochloric acid or 1 N sodium hydroxide as appropriate. This solution was transferred to a clean 1000 mL volumetric flask with the aid of three 100 mL washings of Type 1 water. The solution was diluted to the 1000 mL mark with Type 1 water. The volumetrically correct solution was transferred to a 1 L vessel, purged with nitrogen for one minute and sealed with a nitrogen headspace.

Formulations 6 and 7 were prepared similar to formulations 1-5 except for skipping one or two ingredients as appropriate. For example, EDTA was skipped in preparing formulation 6 whereas BZK and EDTA were skipped in preparing formulation 7.

For formulations 8 and 9, methyl paraben and propyl paraben (in that order) were first dissolved in water before other ingredients were added at the same order as in formulations 1-7.

The order of mixing the ingredients in formulations 1-9 may not have any effect on the stability of the formulations.

Tables 2-4 provide data for the stability of Formulations 1-9 when stored at 2-8° C., at 25° C., at 40° C., and at 50° C. for 1 month, 2 months, 3 months, or 6 months.

For appearance test, the formulations were visually examined at the various time points to determine whether a formulation is clear, colorless, or slightly yellow liquid, and whether there is any particulate matter in the formulation. Characterizations were performed against a matt white background in natural light.

TABLE 2
Appearance test of Formulations 1-9 when stored at 2-8° C., at 25°
C., at 40° C., and at 50° C. for 1 month, 2 months, 3 months, or 6 months.

| | | Visual Observation of the formulations at different times | | | | |
|---|---|---|---|---|---|---|
| | Temp., | (in months) | | | | |
| Ex. # | ° C. | 0 | 1 | 2 | 3 | 6 |
| 1 | 2-8 | Clear & | Clear & | Clear & | Clear & | Clear & |
| | | Colorless | Colorless | Colorless | Colorless | colorless |
| | 25 | Clear & | Clear & | Clear & | Clear & | Clear & |
| | | Colorless | Colorless | Colorless | Colorless | colorless |
| | 40 | Clear & | Clear & | Clear & | Clear & | Clear & |
| | | Colorless | Colorless | Colorless | Colorless | tinted |
| | 50 | Clear & | Clear & | Clear & | Clear & | Clear & |
| | | Colorless | Colorless | Colorless | Colorless | amber |
| 2 | 2-8 | Clear & | Clear & | Clear & | Clear & | Clear & |
| | | Colorless | Colorless | Colorless | Colorless | colorless |
| | 25 | Clear & | Clear & | Clear & | Clear & | Clear & |
| | | Colorless | Colorless | Colorless | Colorless | colorless |
| | 40 | Clear & | Clear & | Clear & | Clear & | Clear & |
| | | Colorless | Colorless | Colorless | Colorless | tinted |
| | 50 | Clear & | Clear & | Clear & | Clear & | Clear & |
| | | Colorless | Colorless | Colorless | Colorless | amber |
| 3 | 2-8 | Clear & | Clear & | Clear & | Clear & | Clear & |
| | | Colorless | Colorless | Colorless | Colorless | colorless |
| | 25 | Clear & | Clear & | Clear & | Clear & | Clear & |
| | | Colorless | Colorless | Colorless | Colorless | colorless |
| | 40 | Clear & | Clear & | Clear & | Clear & | Clear & |
| | | Colorless | Colorless | Colorless | Colorless | colorless |
| | 50 | Clear & | Clear & | Clear & | Clear & | Clear & |
| | | Colorless | Colorless | Colorless | Colorless | amber |
| 4 | 2-8 | Clear & | Clear & | Clear & | Clear & | Clear & |
| | | Colorless | Colorless | Colorless | Colorless | colorless |
| | 25 | Clear & | Clear & | Clear & | Clear & | Clear & |
| | | Colorless | Colorless | Colorless | Colorless | colorless |
| | 40 | Clear & | Clear & | Clear & | Clear & | Clear & |
| | | Colorless | Colorless | Colorless | Colorless | tinted |
| | 50 | Clear & | Clear & | Clear & | Clear & | Clear & |
| | | Colorless | Colorless | Colorless | Colorless | amber |
| 5 | 2-8 | Clear & | Clear & | Clear & | Clear & | Clear & |
| | | Colorless | Colorless | Colorless | Colorless | colorless |
| | 25 | Clear & | Clear & | Clear & | Clear & | Clear & |
| | | Colorless | Colorless | Colorless | Colorless | colorless |
| | 40 | Clear & | Clear & | Clear & | Clear & | Clear & |
| | | Colorless | Colorless | Colorless | Colorless | tinted |
| | 50 | Clear & | Clear & | Clear & | Clear & | Clear & |
| | | Colorless | Colorless | Colorless | Colorless | amber |
| 6 | 2-8 | Clear & | Clear & | Clear & | Clear & | Clear & |
| | | Colorless | Colorless | Colorless | Colorless | colorless |
| | 25 | Clear & | Clear & | Clear & | Clear & | Clear & |
| | | Colorless | Colorless | Colorless | Colorless | colorless |
| | 40 | Clear & | Clear & | Clear & | Clear & | Clear & |
| | | Colorless | Colorless | Colorless | Colorless | tinted |
| | 50 | Clear & | Clear & | Clear & | Clear & pale | Clear & |
| | | Colorless | Colorless | Colorless | yellow | amber |
| 7 | 2-8 | Clear & | Clear & | Clear & | Clear & | Clear & |
| | | Colorless | Colorless | Colorless | Colorless | colorless |
| | 25 | Clear & | Clear & | Clear & | Clear & | Clear & |
| | | Colorless | Colorless | Colorless | Colorless | colorless |
| | 40 | Clear & | Clear & | Clear & | clear & | Clear & |
| | | Colorless | Colorless | Colorless | tinted | tinted |
| | 50 | Clear & | Clear & | Clear & | Clear & pale | Clear & |
| | | Colorless | Colorless | Colorless | yellow | pale yellow |
| 8 | 2-8 | Clear & | Clear & | clear & | clear & | Clear & |
| | | Colorless | Colorless | tinted | tinted | tinted |
| | 25 | Clear & | clear & | Clear & | Clear & pale | Clear & |
| | | Colorless | tinted | pale yellow | yellow | pale yellow |
| | 40 | Clear & | Clear & | Clear & | clear & | Clear & |
| | | Colorless | pale yellow | yellow | yellow | amber |
| | 50 | Clear & | Clear & | Clear & | clear & | Clear & |
| | | Colorless | yellow | amber | amber | amber & |
| | | | | | | dark |
| | | | | | | particulates |
| 9 | 2-8 | Clear & | Clear & | Clear & | clear & | Clear & |
| | | Colorless | Colorless | Colorless | colorless | colorless |
| | 25 | Clear & | Clear & | Clear & | clear & | Clear & |
| | | Colorless | Colorless | Colorless | colorless | tinted |
| | 40 | Clear & | Clear & | Clear & | clear & | Clear & |
| | | Colorless | Colorless | Colorless | tinted | tinted |
| | 50 | Clear & | Clear & | clear & | Clear & pale | Clear & |
| | | Colorless | Colorless | tinted | yellow | amber |

TABLE 3
pH assay of formulations 1-9 at the end of
each storage period at four temperatures.

| | Temp | Time (month) | | | | |
|---|---|---|---|---|---|---|
| Ex. # | (° C.) | 0 | 1 | 2 | 3 | 6 |

| | | | | | | | |
|---|---|---|---|---|---|---|---|
| 1 | 2-8° | C. | 3.5 | 3.5 | 3.5 | 3.6 | 3.5 |
| | 25° | C. | 3.5 | 3.5 | 3.5 | 3.5 | 3.5 |
| | 40° | C. | 3.5 | 3.5 | 3.5 | 3.5 | 3.5 |
| | 50° | C. | 3.5 | 3.5 | 3.6 | 3.5 | 3.5 |
| 2 | 2-8° | C. | 3.5 | 3.5 | 3.5 | 3.5 | 3.4 |
| | 25° | C. | 3.5 | 3.5 | 3.5 | 3.5 | 3.4 |
| | 40° | C. | 3.5 | 3.5 | 3.5 | 3.5 | 3.5 |
| | 50° | C. | 3.5 | 3.5 | 3.5 | 3.5 | 3.4 |
| 3 | 2-8° | C. | 3.5 | 3.5 | 3.5 | 3.5 | 3.5 |
| | 25° | C. | 3.5 | 3.5 | 3.5 | 3.5 | 3.5 |
| | 40° | C. | 3.5 | 3.5 | 3.6 | 3.5 | 3.5 |
| | 50° | C. | 3.5 | 3.5 | 3.5 | 3.5 | 3.5 |
| 4 | 2-8° | C. | 3.5 | 3.5 | 3.5 | 3.5 | 3.5 |
| | 25° | C. | 3.5 | 3.5 | 3.5 | 3.5 | 3.5 |
| | 40° | C. | 3.5 | 3.5 | 3.5 | 3.5 | 3.5 |
| | 50° | C. | 3.5 | 3.5 | 3.5 | 3.5 | 3.5 |
| 5 | 2-8° | C. | 3.5 | 3.5 | 3.5 | 3.5 | 3.5 |
| | 25° | C. | 3.5 | 3.5 | 3.5 | 3.5 | 3.5 |
| | 40° | C. | 3.5 | 3.5 | 3.6 | 3.5 | 3.5 |
| | 50° | C. | 3.5 | 3.5 | 3.5 | 3.5 | 3.5 |
| 6 | 2-8° | C. | 3.5 | 3.5 | 3.5 | 3.6 | 3.5 |
| | 25° | C. | 3.5 | 3.5 | 3.6 | 3.5 | 3.5 |
| | 40° | C. | 3.5 | 3.5 | 3.5 | 3.5 | 3.5 |
| | 50° | C. | 3.5 | 3.5 | 3.6 | 3.5 | 3.5 |
| 7 | 2-8° | C. | 3.5 | 3.5 | 3.5 | 3.5 | 3.5 |
| | 25° | C. | 3.5 | 3.5 | 3.5 | 3.5 | 3.5 |
| | 40° | C. | 3.5 | 3.5 | 3.5 | 3.5 | 3.5 |
| | 50° | C. | 3.5 | 3.5 | 3.6 | 3.5 | 3.4 |
| 8 | 2-8° | C. | 4.0 | 5.1 | 5.1 | 4.9 | 5.0 |
| | 25° | C. | 4.0 | 5.1 | 5.1 | 4.9 | 4.7 |
| | 40° | C. | 4.0 | 4.8 | 4.4 | 4.2 | 3.9 |
| | 50° | C. | 4.0 | 4.3 | 3.9 | 3.7 | 3.4 |
| 9 | 2-8° | C. | 4.0 | 4.6 | 4.5 | 4.5 | 4.5 |
| | 25° | C. | 4.0 | 4.6 | 4.6 | 4.6 | 4.6 |
| | 40° | C. | 4.0 | 4.6 | 4.6 | 4.5 | 4.3 |
| | 50° | C. | 4.0 | 4.6 | 4.3 | 4.1 | 3.7 |

TABLE 4
Naloxone assay of formulations 1-9 after storing them at 2-8° C., at
25° C., at 40° C., and at 50° C. for 1 month, 2 months, 3 months, or 6 months.

| | Temp | | | 0 | 1 | 2 | 3 | 6 |
|---|---|---|---|---|---|---|---|---|
| Ex. # | (° C.) | | Analysis^{#} | Average | Average | Average | Average | Average |
| 1 | 2-8° | C. | Assay (mg/mL) | 10.01 | 10.06 | 10.03 | 10.01 | 10.07 |
| | | | Purity (%) | 99.91 | 99.91 | 99.91 | 99.87 | 99.93 |
| | | | 2,2 Bis (%) | ND | ND | ND | ND | ND |
| | | | Total impurities (%) | 0.09 | 0.09 | 0.09 | 0.13 | 0.07 |
| | 25° | C. | Assay (mg/mL) | 10.01 | 10.01 | 10.06 | 10.03 | 10.09 |
| | | | Purity (%) | 99.91 | 99.90 | 99.87 | 99.86 | 99.89 |
| | | | 2,2 Bis (%) | ND | ND | ND | ND | NQ |
| | | | Total impurities (%) | 0.09 | 0.10 | 0.13 | 0.14 | 0.11 |
| | 40° | C. | Assay (mg/mL) | 10.01 | 10.01 | 10.02 | 10.02 | 10.07 |
| | | | Purity (%) | 99.91 | 99.89 | 99.85 | 99.85 | 99.70 |
| | | | 2,2 Bis (%) | ND | ND | ND | ND | 0.15 |
| | | | Total impurities (%) | 0.09 | 0.11 | 0.15 | 0.15 | 0.30 |
| | 50° | C. | Assay (mg/mL) | 10.01 | 10.06 | 10.06 | 10.05 | 10.04 |
| | | | Purity (%) | 99.91 | 99.84 | 99.73 | 99.69 | 99.32 |
| | | | 2,2 Bis (%) | ND | ND | ND | ND | 0.49 |
| | | | Total impurities (%) | 0.09 | 0.16 | 0.27 | 0.31 | 0.68 |
| 2 | 2-8° | C. | Assay (mg/mL) | 10.02 | 9.99 | 10.02 | 9.98 | 10.06 |
| | | | Purity (%) | 99.91 | 99.90 | 99.92 | 99.86 | 99.89 |
| | | | 2,2 Bis (%) | ND | ND | ND | ND | NQ |
| | | | Total impurities (%) | 0.09 | 0.10 | 0.08 | 0.14 | 0.11 |
| | 25° | C. | Assay (mg/mL) | 10.02 | 10.00 | 10.03 | 10.00 | 10.05 |
| | | | Purity (%) | 99.91 | 99.91 | 99.89 | 99.87 | 99.87 |
| | | | 2,2 Bis (%) | ND | ND | ND | ND | 0.06 |
| | | | Total impurities (%) | 0.09 | 0.09 | 0.11 | 0.13 | 0.13 |
| | 40° | C. | Assay (mg/mL) | 10.02 | 10.02 | 10.08 | 10.01 | 10.03 |
| | | | Purity (%) | 99.91 | 99.89 | 99.85 | 99.85 | 99.68 |
| | | | 2,2 Bis (%) | ND | ND | ND | ND | 0.18 |
| | | | Total impurities (%) | 0.09 | 0.11 | 0.15 | 0.15 | 0.32 |
| | 50° | C. | Assay (mg/mL) | 10.02 | 10.02 | 10.03 | 9.99 | 10.06 |
| | | | Purity (%) | 99.91 | 99.82 | 99.75 | 99.70 | 99.02 |
| | | | 2,2 Bis (%) | ND | ND | ND | ND | 0.78 |
| | | | Total impurities (%) | 0.09 | 0.18 | 0.25 | 0.30 | 0.98 |
| 3 | 2-8° | C. | Assay (mg/mL) | 10.02 | 10.03 | 10.05 | 10.06 | 10.09 |
| | | | Purity (%) | 99.91 | 99.90 | 99.90 | 99.88 | 99.92 |
| | | | 2,2 Bis (%) | ND | ND | ND | ND | 0.02 |
| | | | Total impurities (%) | 0.09 | 0.10 | 0.10 | 0.12 | 0.08 |
| | 25° | C. | Assay (mg/mL) | 10.02 | 10.04 | 10.03 | 10.00 | 10.14 |
| | | | Purity (%) | 99.91 | 99.91 | 99.89 | 99.88 | 99.90 |
| | | | 2,2 Bis (%) | ND | ND | ND | ND | 0.05 |
| | | | Total impurities (%) | 0.09 | 0.09 | 0.11 | 0.12 | 0.10 |
| | 40° | C. | Assay (mg/mL) | 10.02 | 10.02 | 10.03 | 10.04 | 10.05 |
| | | | Purity (%) | 99.91 | 99.87 | 99.84 | 99.84 | 99.76 |
| | | | 2,2 Bis (%) | ND | ND | ND | ND | 0.11 |
| | | | Total impurities (%) | 0.09 | 0.13 | 0.16 | 0.16 | 0.24 |
| | 50° | C. | Assay (mg/mL) | 10.02 | 10.05 | 10.05 | 10.06 | 10.02 |
| | | | Purity (%) | 99.91 | 99.82 | 99.75 | 99.72 | 99.43 |
| | | | 2,2 Bis (%) | ND | ND | ND | ND | 0.37 |
| | | | Total impurities (%) | 0.09 | 0.18 | 0.25 | 0.28 | 0.57 |
| 4 | 2-8° | C. | Assay (mg/mL) | 10.00 | 10.01 | 10.02 | 10.02 | 10.07 |
| | | | Purity (%) | 99.91 | 99.90 | 99.90 | 99.88 | 99.89 |
| | | | 2,2 Bis (%) | ND | ND | ND | ND | 0.04 |
| | | | Total impurities (%) | 0.09 | 0.10 | 0.10 | 0.12 | 0.11 |
| | 25° | C. | Assay (mg/mL) | 10.00 | 10.01 | 10.03 | 10.04 | 10.09 |
| | | | Purity (%) | 99.91 | 99.91 | 99.87 | 99.87 | 99.89 |
| | | | 2,2 Bis (%) | ND | ND | ND | ND | 0.03 |
| | | | Total impurities (%) | 0.09 | 0.09 | 0.13 | 0.13 | 0.11 |
| | 40° | C. | Assay (mg/mL) | 10.00 | 10.04 | 10.00 | 10.05 | 10.10 |
| | | | Purity (%) | 99.91 | 99.89 | 99.86 | 99.85 | 99.69 |
| | | | 2,2 Bis (%) | ND | ND | ND | ND | 0.18 |
| | | | Total impurities (%) | 0.09 | 0.11 | 0.14 | 0.15 | 0.31 |
| | 50° | C. | Assay (mg/mL) | 10.00 | 10.04 | 10.06 | 10.02 | 10.07 |
| | | | Purity (%) | 99.91 | 99.80 | 99.74 | 99.70 | 99.25 |
| | | | 2,2 Bis (%) | ND | ND | ND | ND | 0.56 |
| | | | Total impurities (%) | 0.09 | 0.20 | 0.26 | 0.30 | 0.75 |
| 5 | 2-8° | C. | Assay (mg/mL) | 10.08 | 9.99 | 10.04 | 10.03 | 10.07 |
| | | | Purity (%) | 99.91 | 99.90 | 99.92 | 99.87 | 99.92 |
| | | | 2,2 Bis (%) | ND | ND | ND | ND | 0.08 |
| | | | Total impurities (%) | 0.09 | 0.10 | 0.08 | 0.13 | 0.08 |
| | 25° | C. | Assay (mg/mL) | 10.08 | 10.07 | 10.07 | 9.98 | 10.12 |
| | | | Purity (%) | 99.91 | 99.90 | 99.87 | 99.87 | 99.89 |
| | | | 2,2 Bis (%) | ND | ND | ND | ND | 0.03 |
| | | | Total impurities (%) | 0.09 | 0.10 | 0.13 | 0.13 | 0.11 |
| | 40° | C. | Assay (mg/mL) | 10.08 | 9.99 | 10.04 | 9.98 | 10.12 |
| | | | Purity (%) | 99.91 | 99.88 | 99.87 | 99.84 | 99.79 |
| | | | 2,2 Bis (%) | ND | ND | ND | ND | 0.09 |
| | | | Total impurities (%) | 0.09 | 0.12 | 0.13 | 0.16 | 0.21 |
| | 50° | C. | Assay (mg/mL) | 10.08 | 10.02 | 10.03 | 10.05 | 10.06 |
| | | | Purity (%) | 99.91 | 99.83 | 99.76 | 99.76 | 99.54 |
| | | | 2,2 Bis (%) | ND | ND | ND | ND | 0.28 |
| | | | Total impurities (%) | 0.09 | 0.17 | 0.24 | 0.24 | 0.46 |
| 6 | 2-8° | C. | Assay (mg/mL) | 10.01 | 10.01 | 10.03 | 9.95 | 10.01 |
| | | | Purity (%) | 99.91 | 99.91 | 99.89 | 99.84 | 99.89 |
| | | | 2,2 Bis (%) | ND | ND | ND | ND | NQ |
| | | | Total impurities (%) | 0.09 | 0.09 | 0.11 | 0.16 | 0.11 |
| | 25° | C. | Assay (mg/mL) | 10.01 | 10.05 | 10.03 | 10.01 | 10.03 |
| | | | Purity (%) | 99.91 | 99.90 | 99.86 | 99.86 | 99.85 |
| | | | 2,2 Bis (%) | ND | ND | ND | ND | 0.05 |
| | | | Total impurities (%) | 0.09 | 0.10 | 0.14 | 0.14 | 0.15 |
| | 40° | C. | Assay (mg/mL) | 10.01 | 10.01 | 10.03 | 10.00 | 9.97 |
| | | | Purity (%) | 99.91 | 99.87 | 99.82 | 99.78 | 99.55 |
| | | | 2,2 Bis (%) | ND | ND | ND | ND | 0.24 |
| | | | Total impurities (%) | 0.09 | 0.13 | 0.18 | 0.22 | 0.45 |
| | 50° | C. | Assay (mg/mL) | 10.01 | 10.02 | 10.01 | 9.96 | 9.92 |
| | | | Purity (%) | 99.91 | 99.70 | 99.61 | 99.59 | 99.44 |
| | | | 2,2 Bis (%) | ND | NQ | ND | ND | 0.35 |
| | | | Total impurities (%) | 0.09 | 0.30 | 0.39 | 0.41 | 0.56 |
| 7 | 2-8° | C. | Assay (mg/mL) | 9.99 | 9.95 | 9.98 | 9.94 | 9.95 |
| | | | Purity (%) | 99.91 | 99.90 | 99.89 | 99.85 | 99.90 |
| | | | 2,2 Bis (%) | ND | ND | ND | ND | NQ |
| | | | Total impurities (%) | 0.09 | 0.10 | 0.11 | 0.15 | 0.10 |
| | 25° | C. | Assay (mg/mL) | 9.99 | 9.94 | 10.03 | 9.93 | 10.00 |
| | | | Purity (%) | 99.91 | 99.90 | 99.85 | 99.86 | 99.83 |
| | | | 2,2 Bis (%) | ND | ND | ND | ND | 0.07 |
| | | | Total impurities (%) | 0.09 | 0.10 | 0.15 | 0.14 | 0.17 |
| | 40° | C. | Assay (mg/mL) | 9.99 | 9.96 | 10.02 | 9.90 | 9.91 |
| | | | Purity (%) | 99.91 | 99.86 | 99.81 | 99.76 | 99.50 |
| | | | 2,2 Bis (%) | ND | ND | ND | ND | 0.28 |
| | | | Total impurities (%) | 0.09 | 0.14 | 0.19 | 0.24 | 0.50 |
| | 50° | C. | Assay (mg/mL) | 9.99 | 10.02 | 9.93 | 9.95 | 9.86 |
| | | | Purity (%) | 99.91 | 99.70 | 99.62 | 99.42 | 99.41 |
| | | | 2,2 Bis (%) | ND | ND | ND | ND | 0.35 |
| | | | Total impurities (%) | 0.09 | 0.30 | 0.38 | 0.58 | 0.59 |
| 8 | 2-8° | C. | Assay (mg/mL) | 9.97 | 9.95 | 9.95 | 9.90 | 10.00 |
| | | | Purity (%) | 99.90 | 99.88 | 99.91 | 99.89 | 99.89 |
| | | | 2,2 Bis (%) | ND | ND | ND | ND | ND |
| | | | Total impurities (%) | 0.10 | 0.12 | 0.09 | 0.11 | 0.11 |
| | 25° | C. | Assay (mg/mL) | 9.97 | 9.95 | 9.93 | 9.89 | 9.93 |
| | | | Purity (%) | 99.90 | 99.83 | 99.78 | 99.86 | 99.78 |
| | | | 2,2 Bis (%) | ND | ND | ND | ND | ND |
| | | | Total impurities (%) | 0.10 | 0.17 | 0.22 | 0.14 | 0.22 |
| | 40° | C. | Assay (mg/mL) | 9.97 | 9.93 | 9.86 | 9.79 | 9.77 |
| | | | Purity (%) | 99.90 | 99.73 | 99.59 | 99.68 | 99.67 |
| | | | 2,2 Bis (%) | ND | ND | ND | ND | ND |
| | | | Total impurities (%) | 0.10 | 0.27 | 0.41 | 0.32 | 0.33 |
| | 50° | C. | Assay (mg/mL) | 9.97 | 9.85 | 9.74 | 9.70 | 9.56 |
| | | | Purity (%) | 99.90 | 99.36 | 99.38 | 99.44 | 99.60 |
| | | | 2,2 Bis (%) | ND | ND | ND | ND | ND |
| | | | Total impurities (%) | 0.10 | 0.64 | 0.62 | 0.56 | 0.40 |
| 9 | 2-8° | C. | Assay (mg/mL) | 9.96 | 9.92 | 9.97 | 9.91 | 9.96 |
| | | | Purity (%) | 99.90 | 99.90 | 99.91 | 99.90 | 99.94 |
| | | | 2,2 Bis (%) | ND | ND | ND | ND | ND |
| | | | Total impurities (%) | 0.10 | 0.10 | 0.09 | 0.10 | 0.06 |
| | 25° | C. | Assay (mg/mL) | 9.96 | 9.97 | 9.89 | 9.89 | 9.97 |
| | | | Purity (%) | 99.90 | 99.87 | 99.89 | 99.89 | 99.94 |
| | | | 2,2 Bis (%) | ND | ND | ND | ND | ND |
| | | | Total impurities (%) | 0.10 | 0.13 | 0.11 | 0.11 | 0.06 |
| | 40° | C. | Assay (mg/mL) | 9.96 | 9.94 | 9.97 | 9.94 | 9.90 |
| | | | Purity (%) | 99.90 | 99.86 | 99.88 | 99.85 | 99.83 |
| | | | 2,2 Bis (%) | ND | ND | ND | ND | ND |
| | | | Total impurities (%) | 0.10 | 0.14 | 0.12 | 0.15 | 0.17 |
| | 50° | C. | Assay (mg/mL) | 9.96 | 9.93 | 9.87 | 9.87 | 9.87 |
| | | | Purity (%) | 99.90 | 99.78 | 99.73 | 99.73 | 99.74 |
| | | | 2,2 Bis (%) | ND | ND | ND | ND | ND |
| | | | Total impurities (%) | 0.10 | 0.22 | 0.27 | 0.27 | 0.26 |

ND: Not detected
NQ: Not quantitated
^{#}Purity (%) is calculated from the Total Impurities (%).

### Freeze/Thaw Study

Formulations 1 and 5 were subjected to three cycles of freezing and thawing. Freeze/thaw studies evaluate the effect of short-term excursions outside the label storage conditions, which may occur during shipping, transportation and/or storage. Samples were rotated between freezing (−20° C.), ambient (25° C.) and 40° C. Control samples (samples not subjected to freezing and thawing) were also assessed. Samples were tested at the beginning (time zero) and end of the study. Testing included appearance, naloxone assay and impurities, benzalkonium chloride and EDTA. Table 5 provides freeze/thaw experimental results.

TABLE 5
Freeze/Thaw experimental results

| | | Formulation 1 | | | Formulation 5 | | |
|---|---|---|---|---|---|---|---|
| | | | | Exposed | | | Exposed |
| Test | | T0 | Control ¹ | Sample ² | T0 | Control ¹ | Sample ² |
| Assay of Naloxone (mg/ml) | | 10.07 | 10.06 | 10.07 | 10.03 | 10.03 | 10.04 |
| Impurities (%) | 2,2′-bisnaloxone | ND | ND | ND | ND | ND | ND |
| | Total | 0.11 | 0.13 | 0.19 | 0.12 | 0.14 | 0.11 |

| | | | | | | |
|---|---|---|---|---|---|---|
| Assay of Benzalkonium | 0.21 | 0.20 | 0.20 | 0.11 | 0.11 | 0.11 |
| chloride (mg/ml) | | | | | | |
| Assay of EDTA (mg/ml) | 1.00 | 1.01 | 1.01 | 0.50 | 0.51 | 0.50 |
| Assay of Citric acid (mM) | 24.97 | 25.04 | 25.05 | 9.94 | 10.02 | 10.04 |
| pH | 3.50 | 3.43 | 3.45 | 3.50 | 3.45 | 3.46 |

ND: Not Detected; T0: time zero.
¹ Control samples were not exposed to freeze/thaw cycles. Instead, they were kept at 2-8° C. or at 25° C.
² Exposed samples were subjected to freeze/thaw cycles

The freeze/thaw study indicates that the quality attributes of both formulations (appearance, pH, naloxone assay, EDTA assay, benzalkonium chloride, citric acid and impurities) are not impacted upon exposure to three consecutive freezing and thawing cycles.

### Example 3

### Osmolality Studies

Osmolality study of formulations comprising naloxone, NaCl, BZK, EDTA and, citric acid or methyl paraben or propyl paraben or both methyl and propyl paraben). Samples in Table 5 were prepared in accordance with the method described in Example 1.

TABLE 6
Formulations used in osmolality studies

| | | Formulation | | | | | |
|---|---|---|---|---|---|---|---|
| Component | Unit | 2 | 9 | 10 | 11 | 12 | 13 |
| Naloxone HCl | mg/mL | 10 | 10 | 10 | 10 | 10 | 10 |
| NaCl | mg/mL | 9 | 8.35 | 8 | 10 | 7 | 9.5 |
| BZK | mg/mL | 0.2 | 0 | 0.2 | 0.2 | 0 | 0 |
| EDTA | mg/mL | 0.5 | 0.5 | 0.5 | 0.5 | 0.5 | 0.5 |
| Citric acid | mg/mL | 4.81 | 0 | 4.81 | 4.81 | 0 | 0 |
| Methyl paraben | mg/mL | 0 | 1.8 | 0 | 0 | 1.8 | 1.8 |
| Propyl paraben | mg/mL | 0 | 0.2 | 0 | 0 | 0.2 | 0.2 |
| pH | N/A | 3.5 | 4 | 3.5 | 3.5 | 4 | 4 |

| | | |
|---|---|---|
| Water | N/A | Add water to 1 mL |

N/A = not applicable

The osmolality determination was performed using a Gonotec Osmomat 030 Osmometer. The instrument was calibrated immediately prior to use with a water blank and a 500 mOsmol/kg osmolality standard. To demonstrate the calibration of the instrument, a 300 mOsmol/kg calibration standard was analyzed. The osmolality of each sample was measured three time and the results are summarized below.

The appearance of formulations 2 and 9-13 was characterized by visual examination, i.e. clear, and colorless or slightly yellow liquid, and absence of particulate matter. Characterization was performed against a matt white background in natural light.

Table 7 summarizes the results.

TABLE. 7
Osmolality Results

| | Formulation | | | | | |
|---|---|---|---|---|---|---|
| | 2 | 9 | 10 | 11 | 12 | 13 |
| Appearance | Clear and | Clear and | Clear and | Clear and | Clear and | Clear and |
| | colorless | colorless | colorless | colorless | colorless | colorless |
| mOsm/kg | 386 | 332 | 355 | 417 | 288 | 367 |

Values were averaged from three trials.

All formulations displayed a consistently clear appearance against a matt white background in natural light and with no observed particulate matter.

Formulations 2, 10, and 11 (i.e., formulations containing BZK and citric acid) and formulations 9, 12, and 13 (i.e., formulations containing methyl and propyl parabens) display a clear linear trend which correlates to the concentration of NaCl in each formulation. It should be noted that there is a positive offset on the y intercept (osmolality axis) which is due to the presence of the molecules from the other formulation excipients which are at a constant in each solution.

Formulations 2, 10, and 11 have greater than 300 mOsm/Kg osmolality such that they may be classified as hypertonic. However, the osmolality of formulations 10, 2, and 11 is significantly lower than the generally accepted upper limit of 900 mOsm/Kg for injectable formulations. When osmolality exceeds 900 mOsm/kg, the ability of the peripheral veins to dilute parenteral infusions sufficiently may be compromised causing chemical irritation of the vein.

Formulations 9 and 13 are also hypertonic, whereas Formulation 12, with a value of 288 mOsm/Kg, is isotonic.

From the osmolality tests, it can be concluded that the osmolality of the formulations 1-13 are well within the acceptable range for use as injectable formulations and should not require any reformulation to meet osmolality specification.

### Example 4

### Pharmacokinetics

A phase I, single dose, open label, randomized, three-period crossover study is performed to compare the pharmacokinetics, safety, and tolerability of naloxone administration using the formulations described herein in healthy adults. The study compares the pharmacokinetics of naloxone when the formulations of the present disclosure (0.5 mL to 2 mL at about 7 mg/mL to about 15 mg/mL dosage strengths) are administered, compared to a currently FDA approved naloxone products.

The primary outcome measurements are: (1) plasma concentration time profiles and “area under the curve” (AUC); (2) maximum serum concentration (Cₘₐₓ); (3) time to maximum serum concentration (Tₘₐₓ); (4) elimination rate constant (Kₑₗ); and (5) terminal half-life (t_{½}).

Blood is collected in sodium heparin containing tubes for naloxone PK prior to dosing and 2.5, 5, 10, 15, 20, 30, 45, 60, 120, 180, 240, 300, 360, and 480 minutes after the start of study drug administration. Plasma is separated from whole blood and stored frozen at ≤−20° C. until assayed. Naloxone plasma concentrations are determined by liquid chromatography with tandem mass spectrometry. Conjugated naloxone plasma concentrations may also be determined.

The secondary outcome measurements are: (1) number of subjects with adverse effects; (2) physical examination of subjects; (3) vital signs; and (4) electrocardiograms. Heart rate, blood pressure, and respiration rate are recorded before naloxone dosing and at approximately 30, 60, 120, and 480 minutes after dosing. A 12-lead ECG is obtained prior to and approximately 60 and 480 minutes after each naloxone dose. ECG and vital signs are measured within the 10 minute period before the nominal time for blood collections. Adverse events (AEs) are recorded from the start of study drug administration until clinic discharge. AEs are recorded relative to each dosing session to attempt to establish a relationship between the AE and type of naloxone dose administered.

The data analysis plan examines non-compartmental PK parameters including Cₘₐₓ, Tₘₐₓ, AUC_{0-∞}, AUC₀₋ₜ, t_{½}, λ_{z}, and apparent clearance (CL/F). Pharmacokinetic parameters (Cₘₐₓ, Tₘₐₓ, and AUCs) for IN naloxone are compared with those for the reference IN naloxone. Tₘₐₓ is measured from the time of administration. Dose adjusted values for AUCs and Cₘₐₓ are calculated. The 90% confidence intervals for the ratio (IN/reference IN) of the geometric least squares means of AUC and Cₘₐₓ parameters are constructed for comparison of each treatment. These 90% confidence intervals are obtained by exponentiation of the 90% confidence intervals for the difference between the least squares means based upon an In scale.

AEs are coded using the most recent version of the Medical Dictionary for Regulatory Activities (MedDRA) preferred terms and will be grouped by system, organ, class (SOC) designation. The severity, frequency, and relationship of AEs to study drug are presented by preferred term by SOC grouping. Separate summaries are provided for the study periods: after the administration of each dose of study drug up until the time of the next dose of study drug or clinic discharge. Listings of each individual AE including start date, stop date, severity, relationship, outcome, and duration are provided.

Vital signs, ECG, and clinical laboratory parameters are presented as summary statistics and changes from baseline (with baseline being the measurement prior to each dose).

### Example 5

### Pharmacokinetics of Naloxone on Beagle Dog

Pharmacokinetic (“PK”) data was collected from male and female Beagle dogs. Two (2) animals of each sex were assigned to three cohorts. Each animal was administered formulation 1 or formulation 3 via intramuscular (IM) or intravenous (IV) injection on Days 1, 5, and 9 with a 96-hour washout period in between the dosing events. Each injection was administered at a dose of 0.3 mg/kg. A summary of the exemplary study design is provided in Table 8.

TABLE 8
Experimental Design

| | Study | Study | Study | Animal |
|---|---|---|---|---|
| Cohort | Day 1 | Day 5 | Day 9 | ID |
| 1 | Formulation | Formulation | Formulation | 101-102 (M). |
| | 1 (IM) | 1 (IV) | 3 (IM) | 151-152 (F) |
| 2 | Formulation | Formulation | Formulation | 101-102 (M). |
| | 3 (IM) | 1 (IM) | 1 (IV) | 151-152 (F) |
| 3 | Formulation | Formulation | Formulation | 101-102 (M). |
| | 1 (IV) | 3 (IM) | 1 (IM) | 151-152 (F) |

Table 9 summarizes the doses used in the exemplary study.

TABLE 9
Summary of Doses

| | Dose | Dose Volume | Concentration | HED |
|---|---|---|---|---|
| Formulation | (mg/kg) | (mL/kg) | (mg/mL) | (mg) |
| Developmental | 0.3 | 0.03 | 10 | 10 |
| Formulation 1 (IM) | | | | |
| Developmental | 0.3 | 0.03 | 10 | 10 |
| Formulation 1 (IV) | | | | |
| Developmental | 0.3 | 0.03 | 10 | 10 |
| Formulation 3 (IM) | | | | |

HED—human equivalent dose; the HED is a body surface area conversion between man and other species. The dose in dogs (mg/kg is calculated by dividing the human dose (mg) by an average body weight of 60 kg then multiplying by 1.8 as recommended by “U.S. Food and Drug Administration. 2005. Guidance for Industry. Estimating the Maximum Safe Starting Dose in Initial Clinical Trials for Therapeutics in Adult Healthy Volunteers, Final Rule.”

Blood samples were collected from each animal following IV injections at target time points of pre-dose, and at 0.033, 0.083, 0.167, 0.333, 0.5, 1, 2, 4, and 8 hr. Blood samples were also collected from each animal following IM injections at target time points of pre dose, and at 0.083, 0.167, 0.25, 0.333, 0.5, 1, 2, 4, and 8 hr. Blood samples were collected into tubes containing dipotassium ethylenediaminetetraacetic acid (K2EDTA), processed to plasma, and stored until analyzed for naloxone.

PK Analysis

PK analysis was performed using the target dose (mg/kg), sample collection time points (hr), and the measured concentrations of naloxone (ng/mL) in serum. If the actual sample collection time points were within 5% of target from 0 to 4 hours or within 15 minutes after 4 hours, then the target time points were used. Actual times were used in nine instances. The lower limit of quantitation (LLOQ) in plasma for this method is 0.2 ng/mL.

Plasma analysis followed the method described in “Development and validation of a sensitive LC/MS/MS method for the simultaneous determination of naloxone and its metabolites in mouse plasma,” Journal of Chromatography B, 879 (2011) 2663-2668, Jiang, Wang, Shet, Zhang, Zenke, and Fast. The method utilized protein precipitation in a 96-well format and LC-MS/MS analysis. The literature method was modified to utilize a larger sample size (50 μL) analysis.

TABLE 10
LC-MS System

| Mass Spectrometry System | Sciex (Toronto, Ontario Canada) API 5500 | |
|---|---|---|
| HPLC System | Shimadzu (Kyoto, Japan) Nexera Series | |
| Data Acquisition Software | Analyst, Version 1.7.1 | |
| Ionization Source | Positive Ion Mode | |
| Guard Column | C18, 4 × 2 mm | |
| Analytical Column | C18, 5 μM, 50 × 2.1 mm | |
| Auto-Sampler Temperature | 8° C. | |
| Mobile Phase A | 0.1% Formic acid in water | |
| Mobile Phase B | 0.1% Formic acid in acetonitrile | |
| Flow Rate | 1 mL/minute | |
| | Time (minutes) | % B |
| Mobile Phase Gradient | 0.0 | 2 |
| | 0.2 | 2 |
| | 1.2 | 75 |
| | 1.9 | 75 |
| | 2.0 | 2 |
| | 2.5 | 2 |

| | |
|---|---|
| Injection Volume | 15 μL |
| Run Time | 2.5 minutes |
| Multiple Reaction | Naloxone 328 > 212 amu |
| Monitoring (MRM) | Naloxone-D5 (IS) 333 > 212 amu |

The concentration-time profiles were evaluated using the non-compartmental analysis (NCA) module in the WinNonlin® software program (Version 8.2). For a given concentration versus time profile, the values that were interpreted to define the terminal linear phase were identified by the WinNonlin algorithm to estimate the first order rate constant associated with the terminal linear phase (Lambda z or λz) by performing a regression of the natural logarithm of the concentration over the specified time range.

The concentration-time profiles for the NCA were constructed using the individual serum concentrations of naloxone per time point. If a concentration-time profile did not have at least three time points with measurable concentrations, NCA was not performed. The concentration-time profiles were not considered to be adequately characterized unless the following criteria were met: (1) the coefficient of determination (r2) for the terminal linear phase was ≥0.85, (2) the time of the last observed concentration was greater than three times the half-life, and (3) AUC^{∞} had <20 percent of the area extrapolated.

Results

All concentration and PK parameters were reported to three significant figures.

Naloxone

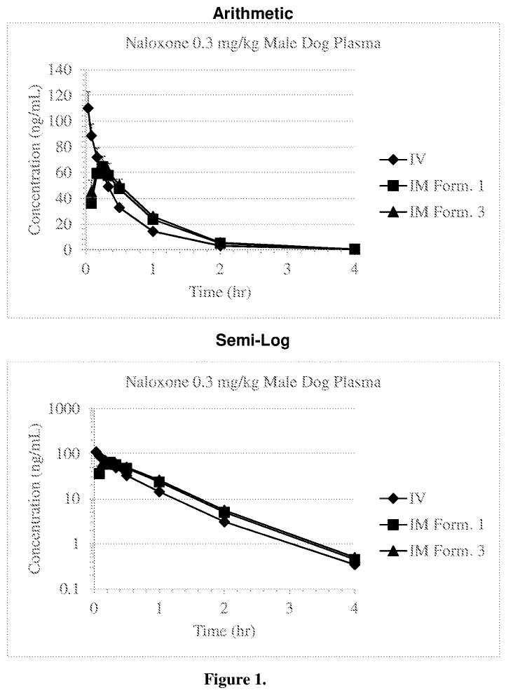

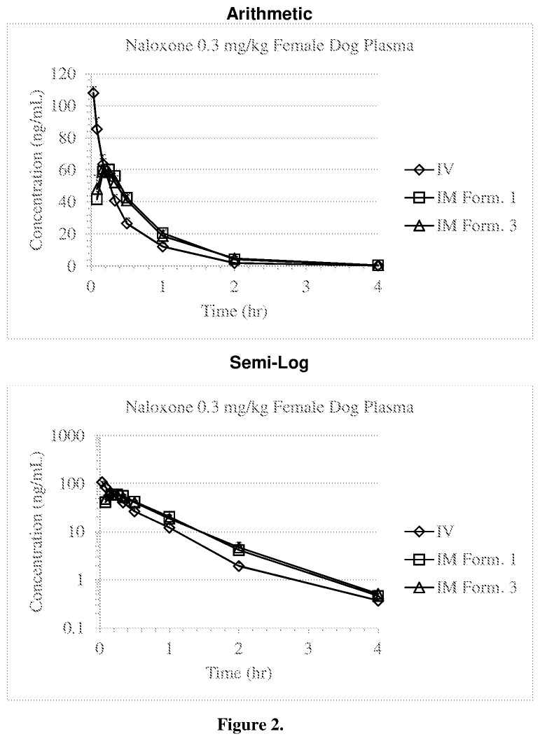

All pre-dose samples were below the limit of quantitation (BLOQ). Table 11 summarizes the PK parameters for naloxone following IV injection and Table 12 summarizes the PK parameters for naloxone following IM injection. Table 13 and Table 14 summarize the partial AUC values. FIGS. 1 and 2 illustrate the group mean male and female naloxone plasma concentrations over time.

The shape of the plasma concentration-time curves for naloxone showed a brief absorption period following IM injection through approximately 10 to 20 minutes. Both IM and IV profiles showed an apparent monophasic decline through the terminal elimination phase. All the profiles passed all three acceptance criteria to adequately characterize the plasma concentration-time profiles.

TABLE 11
PK Parameters for Naloxone Following IV Injection

| | | | | | Elimination | | Volume of | | |
|---|---|---|---|---|---|---|---|---|---|
| | | Animal | Cₘₐₓ | Tₘₐₓ | Half-Life | Clearance | Distribution | AUCₗₐₛₜ | AUC_{∞} |
| Formulation | Sex | ID | (ng/mL) | (hr) | (hr) | (mL/hr/kg) | (mL/kg) | (hr*ng/mL) | (hr*ng/mL) |
| 1 | Male | 101 | 116 | 0.0500 | 0.483 | 6320 | 4410 | 47.3 | 47.4 |
| | | 102 | 153 | 0.0333 | 0.541 | 3910 | 3050 | 76.3 | 76.7 |
| | | 201 | 58.3 | 0.117 | 0.566 | 7170 | 5860 | 41.6 | 41.8 |
| | | 202 | 89.7 | 0.0333 | 0.527 | 6360 | 4840 | 47.0 | 47.2 |
| | | 301 | 118 | 0.0333 | 0.524 | 4250 | 3220 | 70.2 | 70.5 |
| | | 302 | 126 | 0.0333 | 0.529 | 5120 | 3910 | 58.4 | 58.6 |
| | | Mean | 110 | 0.0500 | 0.528 | 5520 | 4210 | 56.8 | 57.0 |
| | | SE | 13 | 0.0137 | 0.011 | 530 | 430 | 5.7 | 5.7 |
| | Female | 151 | 103 | 0.0500 | 0.482 | 5750 | 3990 | 52.1 | 52.2 |
| | | 152 | 122 | 0.0333 | 0.383 | 8440 | 4670 | 34.9 | 35.5 |
| | | 251 | 120 | 0.0333 | 0.590 | 5990 | 5100 | 49.7 | 50.1 |
| | | 252 | 95.8 | 0.0333 | 0.411 | 8600 | 5100 | 34.1 | 34.9 |
| | | 351 | 113 | 0.0833 | 0.515 | 5430 | 4030 | 55.1 | 55.2 |
| | | 352 | 99.9 | 0.0333 | 0.488 | 4790 | 3380 | 62.4 | 62.6 |
| | | Mean | 109 | 0.0444 | 0.478 | 6500 | 4380 | 48.0 | 48.4 |
| | | SE | 4 | 0.0082 | 0.030 | 660 | 280 | 4.6 | 4.5 |

TABLE 2
PK Parameters for Naloxone Following IM Injection

| | | | | | | | Apparent | | | |
|---|---|---|---|---|---|---|---|---|---|---|
| | | | | | Elimination | Apparent | Volume of | | | |
| | | Animal | Cₘₐₓ | Tₘₐₓ | Half-Life | Clearance | Distribution | AUCₗₐₛₜ | AUC_{∞} | Bio- |
| Formulation | Sex | ID | (ng/ mL) | (hr) | (hr) | (mL/hr/kg) | (mL/kg) | (hr*ng/mL) | (hr*ng/mL) | availability |
| 1 | Male | 101 | 46.9 | 0.250 | 0.501 | 6410 | 4630 | 46.6 | 46.8 | 98.7 |
| | | 102 | 71.5 | 0.250 | 0.488 | 4200 | 2960 | 71.1 | 71.4 | 93.1 |
| | | 201 | 69.4 | 0.333 | 0.534 | 4300 | 3310 | 69.3 | 69.7 | 167 |
| | | 202 | 75.2 | 0.250 | 0.532 | 4550 | 3490 | 65.6 | 66.0 | 140 |
| | | 301 | 57.0 | 0.250 | 0.524 | 5390 | 4070 | 55.3 | 55.7 | 79.0 |
| | | 302 | 69.1 | 0.167 | 0.511 | 4640 | 3420 | 64.3 | 64.6 | 110 |
| | | Mean | 64.9 | 0.250 | 0.515 | 4920 | 3650 | 62.0 | 62.3 | 115 |
| | | SE | 4.4 | 0.021 | 0.007 | 340 | 250 | 3.8 | 3.8 | 13 |
| | Female | 151 | 62.0 | 0.167 | 0.495 | 6070 | 4340 | 49.2 | 49.4 | 94.6 |
| | | 152 | 50.2 | 0.250 | 0.490 | 7100 | 5020 | 42.1 | 42.3 | 119 |
| | | 251 | 72.0 | 0.167 | 0.897 | 3980 | 5160 | 75.2 | 75.3 | 150 |
| | | 252 | 57.5 | 0.250 | 0.489 | 5530 | 3900 | 54.1 | 54.3 | 156 |
| | | 351 | 64.3 | 0.333 | 0.547 | 4780 | 3770 | 62.4 | 62.8 | 114 |
| | | 352 | 64.5 | 0.167 | 0.548 | 4950 | 3920 | 60.2 | 60.6 | 96.8 |
| | | Mean | 61.8 | 0.222 | 0.578 | 5400 | 4350 | 57.2 | 57.4 | 122 |
| | | SE | 3.0 | 0.028 | 0.065 | 450 | 250 | 4.7 | 4.7 | 11 |
| 3 | Male | 101 | 35.0 | 0.167 | 0.477 | 6530 | 4490 | 45.8 | 46.0 | 97.0 |
| | | 102 | 70.0 | 0.333 | 0.510 | 3880 | 2850 | 77.0 | 77.4 | 101 |
| | | 201 | 72.8 | 0.267 | 0.489 | 4310 | 3040 | 69.4 | 69.6 | 167 |
| | | 202 | 88.4 | 0.167 | 0.613 | 4370 | 3870 | 68.1 | 68.7 | 146 |
| | | 301 | 56.2 | 0.333 | 0.583 | 4600 | 3870 | 64.5 | 65.2 | 92.5 |
| | | 302 | 87.2 | 0.250 | 0.487 | 3890 | 2730 | 76.9 | 77.1 | 132 |
| | | Mean | 68.3 | 0.253 | 0.527 | 4600 | 3480 | 66.9 | 67.3 | 123 |
| | | SE | 8.3 | 0.030 | 0.023 | 400 | 290 | 4.7 | 4.7 | 12 |
| | Female | 151 | 49.9 | 0.167 | 0.484 | 7660 | 5350 | 39.0 | 39.2 | 75.1 |
| | | 152 | 41.6 | 0.250 | 0.436 | 7740 | 4870 | 36.9 | 38.8 | 109 |
| | | 251 | 79.5 | 0.167 | 0.558 | 3500 | 2820 | 85.0 | 85.6 | 171 |
| | | 252 | 60.3 | 0.250 | 0.363 | 6860 | 3590 | 42.7 | 43.7 | 125 |
| | | 351 | 61.1 | 0.250 | 0.553 | 4610 | 3680 | 64.6 | 65.0 | 118 |
| | | 352 | 76.7 | 0.167 | 0.627 | 4990 | 4510 | 59.7 | 60.2 | 96.2 |
| | | Mean | 61.5 | 0.209 | 0.503 | 5890 | 4140 | 54.7 | 55.4 | 116 |
| | | SE | 6.0 | 0.019 | 0.039 | 720 | 380 | 7.6 | 7.5 | 13 |

TABLE 13
Partial AUC Values for Naloxone Following IV Injection

| | | Animal | AUC_{0-0.0333} | AUC_{0.0333-0.0833} | AUC_{0.0833-0.167} | AUC_{0.167-0.333} | AUC_{0.333-0.5} |
|---|---|---|---|---|---|---|---|
| Formulation | Sex | ID | (hr*ng/mL) | (hr*ng/mL) | (hr*ng/mL) | (hr*ng/mL) | (hr*ng/mL) |
| 1 | Male | 101 | 6.07 | 5.42 | 5.52 | 7.72 | 5.59 |
| | | 102 | 5.51 | 6.88 | 8.93 | 12.9 | 8.50 |
| | | 201 | 1.92 | 2.89 | 4.75 | 8.04 | 5.61 |
| | | 202 | 3.13 | 4.19 | 5.73 | 8.18 | 5.69 |
| | | 301 | 4.25 | 5.31 | 7.81 | 12.7 | 8.72 |
| | | 302 | 4.50 | 5.73 | 7.65 | 10.7 | 6.87 |
| | | Mean | 4.23 | 5.07 | 6.73 | 10.0 | 6.83 |
| | | SE | 0.62 | 0.56 | 0.66 | 1.0 | 0.60 |
| | Female | 151 | 4.20 | 4.95 | 6.10 | 8.29 | 5.58 |
| | | 152 | 5.17 | 4.64 | 4.67 | 6.27 | 3.86 |
| | | 251 | 4.59 | 5.03 | 6.05 | 8.62 | 5.64 |
| | | 252 | 3.49 | 4.24 | 5.15 | 6.96 | 4.30 |
| | | 351 | 3.63 | 5.45 | 8.33 | 11.3 | 6.64 |
| | | 352 | 3.36 | 4.91 | 7.13 | 10.5 | 7.76 |
| | | Mean | 4.07 | 4.87 | 6.24 | 8.66 | 5.63 |
| | | SE | 0.29 | 0.17 | 0.55 | 0.80 | 0.59 |

| | | Animal | AUC_{0.5-1} | AUC₁₋₂ | AUC₂₋₄ | AUC₄₋₈ |
|---|---|---|---|---|---|---|
| Formulation | Sex | ID | (hr*ng/mL) | (hr*ng/mL) | (hr*ng/mL) | (hr*ng/mL) |
| 1 | Male | 101 | 9.64 | 5.47 | 1.88 | 0.187 |
| | | 102 | 14.8 | 13.2 | 5.63 | 0.340 |
| | | 201 | 8.73 | 6.88 | 2.78 | 0.251 |
| | | 202 | 10.1 | 7.13 | 2.77 | 0.229 |
| | | 301 | 15.6 | 11.4 | 4.43 | 0.339 |
| | | 302 | 11.7 | 8.11 | 3.16 | 0.188 |
| | | Mean | 11.8 | 8.69 | 3.44 | 0.256 |
| | | SE | 1.2 | 1.21 | 0.55 | 0.028 |
| | Female | 151 | 11.3 | 9.59 | 2.08 | 0.198 |
| | | 152 | 6.31 | 3.97 | 0.642 | 0.0174 |
| | | 251 | 9.73 | 7.06 | 3.03 | 0.415 |
| | | 252 | 5.92 | 4.06 | 0.759 | 0.0267 |
| | | 351 | 10.4 | 7.06 | 2.27 | 0.180 |
| | | 352 | 14.6 | 10.8 | 3.29 | 0.273 |
| | | Mean | 9.71 | 7.09 | 2.01 | 0.185 |
| | | SE | 1.33 | 1.14 | 0.45 | 0.062 |

TABLE 14
Partial AUC Values for Naloxone Following IM Injection

| | | Animal | AUC_{0-0.0333} | AUC_{0.0333-0.0833} | AUC_{0.0833-0.167} | AUC_{0.167-0.333} | AUC_{0.333-0.5} |
|---|---|---|---|---|---|---|---|
| Formulation | Sex | ID | (hr*ng/mL) | (hr*ng/mL) | (hr*ng/mL) | (hr*ng/mL) | (hr*ng/mL) |
| 1 | Male | 101 | 1.47 | 3.43 | 3.88 | 3.87 | 7.28 |
| | | 102 | 1.66 | 4.44 | 5.71 | 5.69 | 10.3 |
| | | 201 | 1.55 | 3.97 | 5.16 | 5.69 | 10.6 |
| | | 202 | 1.72 | 4.51 | 5.88 | 5.72 | 9.19 |
| | | 301 | 0.845 | 2.92 | 4.42 | 4.28 | 7.01 |
| | | 302 | 1.79 | 4.69 | 5.70 | 5.19 | 8.31 |
| | | Mean | 1.51 | 3.99 | 5.13 | 5.07 | 8.79 |
| | | SE | 0.14 | 0.28 | 0.33 | 0.33 | 0.62 |
| | Female | 151 | 1.69 | 4.29 | 4.93 | 4.58 | 7.74 |
| | | 152 | 0.808 | 2.63 | 3.89 | 3.98 | 6.56 |
| | | 251 | 2.59 | 5.62 | 5.89 | 5.69 | 9.86 |
| | | 252 | 1.18 | 3.25 | 4.43 | 4.53 | 8.10 |
| | | 351 | 1.76 | 4.36 | 5.17 | 5.27 | 9.10 |
| | | 352 | 2.39 | 5.1 | 5.31 | 4.92 | 8.16 |
| | | Mean | 1.74 | 4.21 | 4.94 | 4.83 | 8.25 |
| | | SE | 0.28 | 0.46 | 0.29 | 0.25 | 0.46 |
| 3 | Male | 1.47 | 0.733 | 2.20 | 2.79 | 2.71 | 5.27 |
| | | 1.66 | 2.19 | 4.74 | 5.39 | 5.78 | 10.2 |
| | | 1.55 | 2.43 | 5.76 | 5.91 | 5.77 | 10.2 |
| | | 1.72 | 2.67 | 6.38 | 6.69 | 5.65 | 9.54 |
| | | 0.845 | 0.725 | 1.93 | 3.46 | 4.60 | 8.70 |
| | | 1.79 | 2.00 | 5.23 | 6.81 | 6.83 | 11.7 |
| | | 1.51 | 1.79 | 4.37 | 5.17 | 5.22 | 9.27 |
| | | 0.14 | 0.35 | 0.76 | 0.69 | 0.58 | 0.89 |
| | Female | 1.69 | 1.46 | 3.55 | 3.95 | 3.58 | 5.98 |
| | | 0.808 | 0.75 | 2.49 | 3.45 | 3.41 | 6.39 |
| | | 2.59 | 3.05 | 6.39 | 6.35 | 5.74 | 9.96 |
| | | 1.18 | 2.05 | 4.57 | 5.00 | 4.71 | 7.56 |
| | | 1.76 | 1.78 | 4.14 | 4.87 | 4.74 | 8.10 |
| | | 2.39 | 2.94 | 6.16 | 6.03 | 5.39 | 8.68 |
| | | 1.74 | 2.00 | 4.55 | 4.94 | 4.59 | 7.78 |
| | | 0.28 | 0.36 | 0.62 | 0.46 | 0.38 | 0.60 |

| | | Animal | AUC_{0.5-1} | AUC₁₋₂ | AUC₂₋₄ | AUC₄₋₈ |
|---|---|---|---|---|---|---|
| Formulation | Sex | ID | (hr*ng/mL) | (hr*ng/mL) | (hr*ng/mL) | (hr*ng/mL) |
| 1 | Male | 101 | 14.2 | 9.39 | 3.09 | 0.225 |
| | | 102 | 21.5 | 16.4 | 5.31 | 0.278 |
| | | 201 | 20.9 | 15.6 | 5.78 | 0.443 |
| | | 202 | 17.9 | 14.8 | 5.93 | 0.396 |
| | | 301 | 15.2 | 14.4 | 6.28 | 0.321 |
| | | 302 | 16.9 | 15.4 | 6.43 | 0.306 |
| | | Mean | 17.8 | 14.3 | 5.47 | 0.328 |
| | | SE | 1.2 | 1.0 | 0.50 | 0.032 |
| | Female | 151 | 13.8 | 9.18 | 3.00 | 0.232 |
| | | 152 | 12.0 | 9.00 | 3.25 | 0.174 |
| | | 251 | 19.7 | 17.0 | 6.87 | 2.02 |
| | | 252 | 16.6 | 12.2 | 3.71 | 0.222 |
| | | 351 | 17.0 | 14.2 | 5.52 | 0.415 |
| | | 352 | 15.9 | 12.9 | 5.55 | 0.434 |
| | | Mean | 15.8 | 12.4 | 4.65 | 0.583 |
| | | SE | 1.1 | 1.2 | 0.63 | 0.291 |
| 3 | Male | 1.47 | 13.4 | 14.0 | 4.64 | 0.196 |
| | | 1.66 | 21.6 | 20.1 | 6.99 | 0.364 |
| | | 1.55 | 20.2 | 14.0 | 5.08 | 0.223 |
| | | 1.72 | 18.6 | 12.7 | 5.89 | 0.571 |
| | | 0.845 | 19.2 | 18.0 | 7.89 | 0.647 |
| | | 1.79 | 22.3 | 16.2 | 5.82 | 0.301 |
| | | 1.51 | 19.2 | 15.8 | 6.05 | 0.384 |
| | | 0.14 | 1.3 | 1.1 | 0.49 | 0.076 |
| | Female | 1.69 | 11.0 | 7.55 | 1.95 | 0.166 |
| | | 0.808 | 12.3 | 8.13 | 1.83 | 0.0777 |
| | | 2.59 | 21.6 | 21.0 | 11.0 | 0.539 |
| | | 1.18 | 12.1 | 6.65 | 1.10 | 0.0231 |
| | | 1.76 | 17.7 | 16.5 | 6.87 | 0.444 |
| | | 2.39 | 14.7 | 10.4 | 5.33 | 0.507 |
| | | 1.74 | 14.9 | 11.7 | 4.67 | 0.293 |
| | | 0.28 | 1.7 | 2.4 | 1.56 | 0.094 |

The pharmacokinetic (PK) parameters evaluated for naloxone were Tmax (the time to reach the maximum observed plasma concentration), elimination half-life which is associated with the terminal elimination phase, clearance (IV group) or apparent clearance (IM groups) which is an estimate of the volume of plasma cleared of the analyte over time, and volume of distribution (IV group) or apparent volume of distribution (IM groups) which is an estimate of the apparent volume in which the analyte is distributed. Both IM groups had Tmax values between 0.167 and 0.333 hrs for all animals. Group mean elimination half-life values for males and females were 0.528 and 0.478 hr, respectively, following IV administration, 0.515 and 0.578 hr, respectively, following IM administration of formulation 1, and 0.527 and 0.503 hr, respectively, following IM administration of formulation 3. This suggested no sex or formulation effect. The group mean apparent clearance and apparent volume of distribution were similar when comparing the two IM groups, for both males and females. Additionally, group mean clearance and volume of distribution following IV administration were similar for males and females. These results further suggested no sex or formulation effects.

The systemic exposure parameters evaluated for naloxone were Cmax (the maximum observed plasma concentration), AUClast, and AUC^{∞} (area under the curve from time zero to the last observed time point or extrapolated to infinity). When comparing the two IM groups with the IV groups, group mean AUClast and AUC^{∞} values were similar. The absolute bioavailability for the individual animals that received an IM administration of formulation 1 was greater than 93% for all but one animal. The relative bioavailability for the individual animals that received an IM administration of formulation 3 when compared to the IV group that used formulation 1 was greater than 92% for all but one animal. Comparison of Cmax and AUC values for the two IM groups suggested no formulation effects on systemic exposure of naloxone. Overall, there were no apparent sex effects on naloxone following IV or IM administration. In addition, there were no apparent formulation effects on naloxone following IM administration.

### Summary of the Results

PK parameters were determined and evaluated for naloxone after a single IV of formulation 1, a single IM injection of formulation 1, and a single IM injection of formulation 3.

For naloxone PK analysis following both IM and IV injection, group mean elimination half-life values were short, ranging between approximately 28 to 35 minutes. Following IM injection Tmax values typically occurred around 15 minutes post injection. Overall, the similarities in group mean elimination half-life, clearance, volume of distribution, Cmax and AUC values suggested no apparent sex effects following IV or IM injection. Additionally, when comparing group mean PK and systemic exposure parameters, there were no apparent formulation effects on naloxone following IM injection.

What is claimed is:

1. A formulation comprising:
between about 0.3% (w/v) and about 3.0% (w/v) naloxone or a pharmaceutically acceptable salt thereof,
between about 0.3% (w/v) and about 3% (w/v) NaCl,
between about 0.005% (w/v) and about 0.05% (w/v) BZK,
between about 0.02% (w/v) and about 0.25% (w/v) EDTA, and
between about 0.10% (w/v) and about 1.0% (w/v) citric acid.

2. The formulation of claim 1, comprising:
between about 0.7% (w/v) and about 1.5% (w/v) naloxone or a pharmaceutically acceptable salt thereof,
between about 0.5% (w/v) and about 1.5% (w/v) NaCl,
between about 0.005% (w/v) and about 0.03% (w/v) BZK,
between about 0.05% (w/v) and about 0.10% (w/v) EDTA, and
between about 0.20% (w/v) and about 0.50% (w/v) citric acid.

3. The formulation of claim 2, wherein the formulation comprises about 1% (w/v) naloxone or a pharmaceutically acceptable salt thereof.

4. The formulation of any one of claims 1-3, wherein the NaCl is present in a concentration between about 0.7% (w/v) and about 1.4% (w/v).

5. The formulation of any one of claims 1-3, wherein the NaCl is present in a concentration between about 0.8% (w/v) and about 1.2% (w/v).

6. The formulation of any one of claims 1-3, wherein the NaCl is present in a concentration about 0.8% (w/v), about 0.9% (w/v), or about 1.0% (w/v).

7. The formulation of any one of claims 1-6, wherein BZK is present in an amount between about 0.01% (w/v) and about 0.02% (w/v).

8. The formulation of any one of claims 1-6 wherein BZK is present in an amount about 0.008% (w/v), 0.009% (w/v), 0.010% (w/v), about 0.012% (w/v), about 0.014% (w/v), about 0.016% (w/v), about 0.018% (w/v), about 0.02% (w/v), about 0.021% (w/v), or about 0.022% (w/v).

9. The formulation of any one of claims 1-8, wherein the formulation has an osmolality between about 250 mOsm and 500 mOsm.

10. The formulation of any one of claims 1-9, wherein the formulation has a pH between about 3 and about 7, between about 3 and about 6, between about 3 and about 5, or between about 3 and about 4.

11. The formulation of any one of claims 1-9, wherein the formulation has a pH about 3.5.

12. The formulation of any one of claims 1-11, wherein the EDTA is present about 0.03%, about 0.04%, about 0.05% about 0.06%, about 0.07%, about 0.08%, about 0.09%, about 0.10%, about 0.11%, or about 0.12% (w/v).

13. The formulation of any one of claims 1-12, wherein the formulation does not comprise a methylparaben, a propylparaben, or combinations thereof.

14. The formulation of any one of claims 1-12, wherein the formulation does not comprise alkylparabens.

15. The formulation of any one of claims 1-12, wherein the total amount of an alkylparaben present in the formulation is no greater than about 0.001% (w/v).

16. An auto-injector device, wherein the device comprises a housing, a medicament container, and a delivery member, wherein the medicament container is disposed within the housing and defines an internal volume containing the formulation of any of claims 1-15.

17. A method of treating an opioid exposure in a subject in need thereof, the method comprising:
administering the formulation of any one of claims 1-15, to a subject.

18. The method of claim 17, wherein the administering comprises injecting the subject with the formulation.

19. The method of claim 18, wherein the administration comprises injecting the subject intramuscularly with the formulation.

20. The method of claim 18, wherein the administration comprises injecting the subject subcutaneously with the formulation.

21. The method of claim 18, wherein the administration is performed with an autoinjector.

22. A formulation comprising,
a) between about 0.3% (w/v) and about 3.0% (w/v) naloxone or a pharmaceutically acceptable salt thereof;
b) between about 0.3% (w/v) and about 3% (w/v) NaCl;
c) between about 0.005% (w/v) and about 0.05% (w/v) BZK;
d) between about 0.02% (w/v) and about 0.25% (w/v) EDTA; and
e) between about 0.10% (w/v) and about 1.0% (w/v) citric acid;
wherein administration of about 0.15 mg/Kg to about 0.45 mg/Kg to a subject in need of provides
i. an elimination half-life of between about 20 minutes and about 60 minutes;
ii. an AUC in plasma at between about 40 and about 70 hr*ng/mL when the AUC is measured at three times the elimination half-life or greater;
iii. a Cmax from about 30 to about 150 nanograms per milliliter;
iv. a Tmax between about 10 minutes and about 20 minutes; or
v. bioavailability greater than about 90%; or
vi. any combination thereof.

23. A method of treating an opioid exposure in a subject in need thereof, the method comprising administering to the subject about 0.15 mg/Kg to about 0.45 mg/Kg of naloxone from a formulation comprising:
a) between about 0.3% (w/v) and about 3.0% (w/v) naloxone or a pharmaceutically acceptable salt thereof;
b) between about 0.3% (w/v) and about 3% (w/v) NaCl;
c) between about 0.005% (w/v) and about 0.05% (w/v) BZK;
d) between about 0.02% (w/v) and about 0.25% (w/v) EDTA; and
e) between about 0.10% (w/v) and about 1.0% (w/v) citric acid;
wherein said administration provides:
i. an elimination half-life of between about 20 minutes and about 60 minutes;
ii. an AUC in plasma at between about 40 and about 70 hr*ng/mL when the AUC is measured at three times the elimination half-life or greater;
iii. a Cmax from about 30 to about 150 nanograms per milliliter;
iv. a Tmax between about 10 minutes and about 20 minutes;
v. a bioavailability greater than about 90%; or
vi. a combination thereof.

24. The method of claim 23, wherein said administration comprises an auto-injector device comprising a housing, a medicament container, and a delivery member, wherein the medicament container is disposed within the housing and defines an internal volume containing the formulation.

25. The method of claim 24, wherein said administration provides an elimination half-life of between about 20 minutes and about 60 minutes; and/or an AUC in plasma at between about 40 and about 70 hr*ng/mL when the AUC is measured at three times the elimination half-life or greater.

26. The method of claim 23, wherein said administration provides an elimination half-life of between about 20 minutes and about 60 minutes; and/or a Cmax from about 30 to about 150 nanograms per milliliter.

27. The method of claim 23, wherein said administration provides an elimination half-life of between about 20 minutes and about 60 minutes; and/or a Tmax between about 10 minutes and about 20 minutes.

28. The method of claim 23, wherein said administration provides an AUC in plasma at between about 40 and about 70 hr*ng/mL when the AUC is measured at three times the elimination half-life or greater; and/or a Cmax from about 30 to about 150 nanograms per milliliter.

29. The method of claim 23, wherein said administration provides an AUC in plasma at between about 40 and about 70 hr*ng/mL when the AUC is measured at three times the elimination half-life or greater; and/or a Tmax between about 10 minutes and about 20 minutes.

30. The method of claim 23, wherein said administration provides a Cmax from about 30 to about 150 nanograms per milliliter; and/or a Tmax between about 10 minutes and about 20 minutes.

31. The method of any of claims 23-31, wherein an elimination half-life of between about 20 minutes and about 60 minutes; and/or a bioavailability greater than about 90%.

32. The method of any of claims 23-31, wherein an AUC in plasma at between about 40 and about 70 hr*ng/mL when the AUC is measured at three times the elimination half-life or greater; and/or a bioavailability greater than about 90%.

33. The method of any of claims 23-31, wherein a Cmax from about 30 to about 150 nanograms per milliliter; and/or a bioavailability greater than about 90%.

34. The method of any of claims 23-31, wherein a Tmax between about 10 minutes and about 20 minutes; and/or or a bioavailability greater than about 90%.

35. The method of any of claims 23-34, wherein the formulation has an osmolality between about 250 mOsm and 500 mOsm.

36. The method of any of claims 23-35, wherein the formulation has a pH between about 3 and about 7, between about 3 and about 6, between about 3 and about 5, or between about 3 and about 4.

37. The method of any of claims 23-36, wherein the formulation does not comprise a methylparaben, a propylparaben, or combinations thereof.

38. The method of any of claims 23-37, wherein the subject is administered a dose between about 8 mg and about 12 mg.

39. The method of any of claims 23-38, wherein the method comprises administering to the subject about 0.16 mg/Kg of naloxone from a formulation comprising:
a) about 1.0% (w/v) naloxone or a pharmaceutically acceptable salt thereof;
b) about 0.9% (w/v) NaCl;
c) about 0.02% (w/v) BZK;
d) about 0.1% (w/v) EDTA; and
e) about 0.5% (w/v) citric acid.

40. The method of any of claims 23-38, wherein the method comprises administering to the subject about 0.16 mg/Kg of naloxone from a formulation comprising:
a) about 1.0% (w/v) naloxone or a pharmaceutically acceptable salt thereof;
b) about 0.9% (w/v) NaCl;
c) about 0.01% (w/v) BZK;
d) about 0.1% (w/v) EDTA; and
e) about 0.5% (w/v) citric acid.

41. The method of any of claims 23-40, wherein the subject is administered about 10 mg naloxone.

42. The method of any of claims 23-41, wherein the subject is a human.

43. The method of any of claims 23-39, wherein the method comprises administering to the subject about 0.3 mg/Kg of naloxone from a formulation comprising:
a) about 1.0% (w/v) naloxone or a pharmaceutically acceptable salt thereof;
b) about 0.9% (w/v) NaCl;
c) about 0.02% (w/v) BZK;
d) about 0.1% (w/v) EDTA; and
e) about 0.5% (w/v) citric acid.

44. The method of any of claims 23-38, wherein the method comprises administering to the subject about 0.3 mg/Kg of naloxone from a formulation comprising:
a) about 1.0% (w/v) naloxone or a pharmaceutically acceptable salt thereof;
b) about 0.9% (w/v) NaCl;
c) about 0.01% (w/v) BZK;
d) about 0.1% (w/v) EDTA; and
e) about 0.5% (w/v) citric acid.

45. The method of any of claims 43 and 44, wherein the subject is a canine.
